(19) United States
(12) Patent Application Publication
(10) Pub. No.: US 2002/0169301 A1
(43) Pub. Date: Nov. 14, 2002

(54) Novel bHLH type transcription factor genes DEC2

(76) Inventors: Katsumi Fujimoto, Hiroshima (JP); Mei Shin, Hiroshima (JP); Yukio Kato, Hiroshima (JP)

Correspondence Address: JANIS K. FRASER, PH.D., J.D., Fish & Richardson P.C., 225 Franklin Street, Boston, MA, 02110-2804, US

(21) Appl. No.: 10/078,650

(22) Filed: Feb. 19, 2002

Related U.S. Application Data
(63) Continuation-in-part of application No. PCT/JP00/03991, filed on Jun. 19, 2000.

(30) Foreign Application Priority Data
Aug. 19, 1999 (JP) 11-233286

(51) Int. Cl. C07H 21/02; C07H 21/04

(52) U.S. Cl. 536/23.1

(57) ABSTRACT

Human- and mouse-derived bHLH type transcription factors were isolated. These transcription factors shared homology with the transcription factor DEC1, the expression of which is induced in human cartilage cells when differentiated with cAMP. Thus, these isolated transcription factors are thought to be novel transcription factors belonging to the DEC1 subfamily. The transcription factors of the present invention are also homologous to mouse Stra13 and rat SHARP that are related to neurocyte differentiation. The transcription factors of the present invention may be used as tools for developing pharmaceutical agents for diseases related to the differentiation and proliferation of cells.

### CROSS REFERENCE TO RELATED APPLICATIONS

This application is a continuation-in-part of International Application No. PCT/JP00/03991, filed Jun. 19, 2000, which claims priority to Japanese Patent Application No. 11-233286, filed Aug. 19, 1999. These applications are incorporated herein by reference in their entirety.

### TECHNICAL FIELD

The present invention relates to novel bHLH type transcription factors and genes thereof. These molecules may be used, for example in the development of pharmaceutical agents.

### BACKGROUND

In animals, the development and differentiation are regulated by various transcription factors that control tissue differentiation and cell proliferation. Particularly, bHLH (basic domain-Helix-Loop-Helix structure) type transcription factors are reported to control cell proliferation and differentiation in myogenesis (Weintraub et al., Science 251:761-6, 1991), neurogenesis (Jan et al., Cell 75:827-830, 1993), hematopoiesis (Zhuang et al., Cell 79:875-884, 1994), and such. bHLH type transcription factors are characterized by a conserved region (bHLH structure) of 60 to 70 amino acids, which mediates dimer formation of proteins. The HLH domain is mostly located downstream adjacent to the basic (b) domain necessary for DNA binding. Generally, these proteins bind to DNA by forming homodimers or heterodimers to regulate transcription (Jan et al., Cell 75:827-830, 1993). bHLH transcription factors are classified into several families according to their structural and functional characteristics (Dang et al., Proc. Natl. Acad. Sci. USA 89:559-602, 1992; Ohsako et al., Genes Dev. 8:2743-2755, 1994). Among these, HES family genes, which are mammalian homologues of the Drosophila hairy [h] (Rushlow et al., EMBO J. 8:3095-3103, 1989) and enhancer-of-split [E(spl)] (Klambt et al., EMBO J. 8:203-210, 1989) genes, are related to neurogenesis and are reported to function as repressors (negative regulatory factor) of transcription (Sasai et al., Genes Dev. 6:2620-2634, 1992; Ishibashi et al., Eur. J. Biochem. 215:645-652, 1993; Akazawa et al., J. Biol. Chem. 267:21879-21885, 1992; Ohsako et al., Genes Dev. 8:2743-2755, 1994; Dawson et al., Mol. Cell. Biol. 15:6923-6931, 1995; Jan et al., Cell 75:827-830, 1993).

The inventors previously cloned bHLH type transcription factor DEC1, the expression of which is specifically induced by dibutyryl cAMP in human cultured chondrocytes that retain the characteristics of cartilages, using the subtraction method (Unexamined Published Japanese Patent Application (JP-A) No. Hei 11-75882). DEC1 is homologous to the hairy, enhancer-of-split [E(spl)], and HES family genes (Sasai et al., Genes Dev. 6:2620-2634, 1992; Ishibashi et al., Eur. J. Biochem. 215:645-652, 1993), and is considered to be a novel bHLH type transcription factor phylogenetically related to these transcription factors. Expression of DEC1 is observed in not only chondrocytes, but also other multiple human tissues. Furthermore, the fact that cAMP induces expression of DEC1 mRNA in many cells suggests that DEC1 is related to not only the regulation of chondrocyte differentiation, but also the regulation of differentiation and cell proliferation of other tissues (Shen et al., Biochem. Biophys. Res. Commun. 236:294-298, 1997).

Besides DEC1, mouse Stra13 (Boudjelal et al., Genes Dev. 11:2052-2065, 1997) and rat SHARP (Rossner et al., Mol. Cell. Neurosci. 9:460-475, 1997) are also identified as bHLH type transcription factors that show relatively high homology with h/E(spl)/HES. The expression of mouse Stra13 is induced when embryonal carcinoma cell line P19 is treated with retinoic acid, and a high expression induces differentiation of neurocytes. Moreover, this transcription factor represses transcriptional activation of other transcription factors by forming a heterodimer, functioning as a transcriptional repressor. During embryogenesis, expression of Stra13 is observed in the endoderm and mesoderm, besides the neuroectoderm (Boudjelal et al., Genes Dev. 11:2052-2065, 1997). Rat SHARP is a bHLH type transcription factor whose expression rises in the late phase of embryogenesis and after birth and is expected to function in the plasticity of the brain. Furthermore, its expression is induced by the addition of NGF to PC12 cells, or by the addition of kainic acid in vivo. Two genes, SHARP-2 and SHARP-1, showing different tissue distributions have been identified as rat SHARP genes. Therefore, it is supposed that human DEC1 also forms a subfamily with other analogous genes. The isolation of these unknown bHLH type transcription factors is thought to be an important step in developing new pharmaceutical agents.

### SUMMARY

An objective of the present invention is to provide novel bHLH type transcription factors and genes thereof, as well as methods for producing them, and their uses.

The present inventors searched for human cDNA fragments that are similar to DEC1 in an EST database to clone a novel transcription factor that constitutes a DEC1 subfamily. Using a primer synthesized based on the nucleotide sequence information of a thus obtained cDNA fragment, PCR cloning was conducted using human skeletal muscle cDNA as template. Furthermore, 3′RACE and 5′RACE methods were performed to obtain multiple cDNA, and the full-length cDNA sequences were determined. One of these cDNA with a full-length of 3641 bp encoded a protein of 482 amino acids, the molecular weight of which was estimated to be 50.5 kDa (this clone was named “human DEC2a”). The amino acid sequence of human DEC2a showed the highest similarity to that of rat SHARP-1. Particularly, the N-terminus half was 90% or more identical, whereas the C-terminus half showed a low similarity where only parts of the sequence were identical. This protein was supposed to be a new member of the DEC1 subfamily, because the bHLH region showed a high similarity. Another cDNA with a full-length of 1511 bp encoded a protein of 484 amino acids and was presumed to have a molecular weight of 50.7 kDa. This clone showed significant homology with human DEC2a and thus was named “human DEC2b”.

Moreover, the present inventors succeeded in obtaining the mouse DEC2a cDNA corresponding to the human DEC2a cDNA, which has a full-length of 1421 bp and encodes a protein of 410 amino acids having an estimated molecular weight of 43.9 kDa, by PCR using mouse skeletal muscle cDNA as the template.

The novel bHLH type transcription factors “DEC2” isolated by the present inventors are thought to be members of the DEC1 subfamily. Consequently, they are expected to be useful as novel factors that control development and tissue differentiation. It is also expected that they may be used as markers to determine developmental stages and cell differentiation. Additionally, they are expected to be useful as targets in developing pharmaceutical agents for various diseases associated with the proteins of the present invention.

The present invention relates to novel bHLH type transcription factors and their genes, their production, and uses as well. More specifically, it provides:

(1) a DNA according to any one of the following (a) to (d):

(a) a DNA comprising the coding region of the nucleotide sequence of SEQ ID NO:1, 11, or 13,

(b) a DNA encoding a protein comprising the amino acid sequence of SEQ ID NO:2, 12, or 14,

(c) a DNA encoding a protein comprising an amino acid sequence in which one or more amino acids in the amino acid sequence of SEQ ID NO:2, 12, or 14 has been substituted, deleted, inserted, and/or added, wherein the protein is functionally equivalent to the protein comprising the amino acid sequence of SEQ ID NO:2, 12, or 14, and,

(d) a DNA that hybridizes under stringent conditions to a DNA comprising the nucleotide sequence of SEQ ID NO:1, 11, or 13, and encodes a protein that is functionally equivalent to the protein comprising the amino acid sequence of SEQ ID NO:2, 12, or 14;

(2) a DNA that encodes a partial peptide of the protein of SEQ ID NO:2, 12, or 14;

(3) a vector into which a DNA of (1) or (2) is inserted;

(4) a transformant carrying a DNA of (1) or (2), or the vector of (3);

(5) a protein or peptide encoded by a DNA of (1) or (2);

(6) a method for producing the protein or peptide of (5), comprising the steps of:

culturing the transformant of (4), and,

recovering the expressed protein from the transformant or culture supernatant;

(7) a DNA comprising at least 15 nucleotides, wherein the DNA is complementary to a DNA comprising the nucleotide sequence of SEQ ID NO: 1, 11, or 13, or to the complementary strand thereof;

(8) a method of screening for a compound that binds to the protein of (5), comprising the steps of:

(a) contacting a test sample with said protein or partial peptide,

(b) detecting the binding activity of said protein or partial peptide with the test sample, and,

(c) selecting a compound that has a binding activity to said protein or partial peptide;

(9) a compound that binds to the protein of (5);

(10) the compound of (9), wherein said compound is an antibody; and

(11) the compound of (9), wherein said compound is isolated by a method as set forth in (8).

The present invention provides novel bHLH type transcription factors and DNA encoding the proteins. The nucleotide sequences of the cDNA of bHLH type transcription factors, human DEC2a, human DEC2b, and mouse DEC2a (collectively called “DEC2”) isolated by the present inventors, and the amino acid sequences of the proteins encoded by the cDNA are shown in SEQ ID NO:1, 11, 13, 2, 12, and 14, respectively. The “DEC2” genes isolated by the present inventors are suggested to be related not only to differentiation and proliferation of tissues including cartilages, but also to functions of various adult tissues since they were highly homologous to bHLH type transcription factor SHARP in the bHLH region, which is suggested to be involved in the plasticity of cells of the central nervous system in rats. Thus, “DEC2” proteins of the present invention and DNA encoding these proteins are not only useful as factors that control tissue differentiation and cell function, or as differentiation markers, but they may also be applied to the diagnosis, prophylaxis, and treatment of diseases related to the proteins of the present invention.

For example, “DEC2” is important in elucidating the differentiation and deformation mechanisms of cartilages, and it is also expected to be useful in developing gene therapy methods against osteoarthritis, rheumatoid arthritis, etc.

Furthermore, the present invention includes proteins functionally equivalent to the “DEC2” proteins (SEQ ID NOs:2, 12, and 14). Such proteins include, for example, homologue proteins derived from other organisms, which correspond to the “DEC2” proteins, and mutants of “DEC2” proteins. Herein, “functionally equivalent” means that the target protein has a function of a bHLH type transcription factor. The function of a bHLH type transcription factor is to form a homodimer (or a heterodimer with other bHLH type transcription factors) and have an activity to negatively or positively regulate transcriptional activity. Further, a bHLH type transcription factor may have a binding activity towards CANNTG and/or CACNAG. Methods for measuring the binding activity towards CANNTG and/or CACNAG are well known (for example, Ohsako et al., Genes & Dev. 8:2743-2755, 1994).

One method well known to those skilled in the art for preparing functionally equivalent proteins is to introduce mutations into proteins. For example, one skilled in the art can prepare proteins functionally equivalent to a “DEC2” protein by introducing appropriate mutations into the amino acid sequence of the protein (SEQ ID NO:2, 12, or 14), by using site-specific mutagenesis (Hashimoto-Gotoh et al., Gene 152:271-275, 1995; Zoller et al., Methods Enzymol. 100:468-500, 1983; Kramer et al., Nucleic Acids Res. 12:9441-9456, 1984; Kramer et al., Methods. Enzymol. 154:350-367, 1987; Kunkel, Proc. Natl. Acad. Sci. USA 82:488-492, 1985; Kunkel, Methods Enzymol. 85:2763-2766, 1988), and such. Mutation of amino acids may occur in nature, too. Furthermore, the proteins of the present invention include a protein comprising an amino acid sequence of a “DEC2” protein (SEQ ID NO:2, 12, or 14) in which one or more amino acids have been mutated, which is functionally equivalent to the DEC2 protein. In such a mutant protein, the number of amino acids mutated are considered to be usually 100 residues or less, preferably 50 residues or less, more preferably 20 residues or less, even more preferably 10 residues or less, and still more preferably 5 residues or less.

It is preferable to mutate an amino acid residue into one that allows the properties of the amino acid side-chain to be conserved. Examples of properties of amino acid side chains include chains having: hydrophobic amino acids (A, I, L, M, F, P, W, Y, V), hydrophilic amino acids (R, D, N, C, E, Q, G, H, K, S, T), and amino acids comprising the following side chains: aliphatic side-chains (G, A, V, L, I, P); hydroxyl group-containing side-chains (S, T, Y); sulfur atom-containing side-chains (C, M); carboxylic acid- and amide-containing side-chains (D, N, E, Q); base-containing side-chain (R, K, H); and aromatic-containing side-chains (H, F, Y, W) (The letters within parenthesis indicate the one-letter codes of amino acids).

It is well known that a protein having a deletion, addition, and/or substitution of one or more amino acid residues in the sequence of the protein can retain the original biological activity (Mark et al., Proc. Natl. Acad. Sci. USA 81:5662-5666, 1984; Zoller et al., Nucleic Acids Res. 10:6487-6500, 1982; Wang et al., Science 224:1431-1433; Dalbadie-McFarland et al., Proc. Natl. Acad. Sci. USA 79:6409-6413, 1982).

A protein in which amino acid residues have been added to the amino acid sequence of a “DEC2” protein includes a fusion protein comprising the “DEC2” protein. The present invention includes a fusion protein in which one or more “DEC2” proteins and one or more other proteins or peptides are fused. Methods well known in the art may be used to generate a fusion protein of the present invention. For example, a DNA encoding a “DEC2” protein (SEQ ID NO:2, 12, or 14) and a DNA encoding another protein or peptide are linked in frame and introduced into an expression vector. The fusion protein is then expressed in a host cell. The protein or peptide fused to a protein of the present invention is not limited to any specific protein or peptide.

Known peptides, for example, FLAG (Hopp et al., Biotechnology 6:1204-1210, 1988), 6×His containing six His (histidine) residues, 10×His, Influenza agglutinin (HA), human c-myc fragment, VSP-GP fragment, p18HIV fragment, T7-tag, HSV-tag, E-tag, SV40T antigen fragment, 1ck tag, α-tubulin fragment, B-tag, Protein C fragment, and such, can be used as peptides that are fused to a protein of the present invention. Examples of proteins that are fused to a protein of the invention are, GST (glutathione-S-transferase), Influenza agglutinin (HA), immunoglobulin constant region, β-galactosidase, MBP (maltose-binding protein), and such.

Fusion proteins can be prepared by fusing commercially available DNA encoding these peptides or proteins with a DNA encoding a protein of the present invention and expressing the fused DNA prepared.

An alternative method known to those skilled in the art for preparing functionally equivalent proteins is, for example, the method utilizing the hybridization technique (Sambrook. et al., Molecular Cloning 2nd ed. 9.47-9.58, Cold Spring Harbor Lab. Press, 1989). Generally, one skilled in the art can isolate DNA highly homologous to the whole or part of a DNA sequence encoding a “DEC2” protein (SEQ ID NO:1, 11, or 13), and then isolate a protein functionally equivalent to the “DEC2” protein from those DNA isolated. The proteins of the present invention thus include proteins encoded by DNA that hybridize with the whole or part of a DNA sequence encoding a “DEC2” protein under stringent conditions, in which the proteins are functionally equivalent to the “DEC2” protein. These proteins include mammalian homologues (e.g., proteins encoded by genes of humans, monkeys, mice, rats, rabbits, cattle, pigs, dogs, and cats). In isolating cDNA highly homologous to a DNA encoding a “DEC2” protein from animals, it may be preferable to use tissues such as brain, skeletal muscle, testis, placenta, large intestine, spleen, cartilage, and so on.

Hybridization conditions for isolating a DNA encoding a protein functionally equivalent to a “DEC2” protein may be appropriately selected by a person skilled in the art. A hybridization conducted under stringent conditions is one conducted, for example, in a solution of 6×SSC (0.9 M sodium chloride, 0.09 M sodium citrate), 0.5% SDS, 10 mM EDTA, 5×Denhardt's solution (0.1% (w/v) Ficoll, 0.1% (w/v) polyvinylpyrrolidone), 0.1% (w/v) BSA), 10 mg/ml denatured salmon sperm DNA at 60° C. A more preferable stringent condition is conducting hybridization at 68° C. in the solution above. However, other than temperature, several factors, such as salt concentration, can influence the stringency of hybridization and one skilled in the art can suitably select the factors to accomplish a similar stringency.

In place of hybridization, a gene amplification method using primers synthesized based on the sequence information of the DNA (SEQ ID NO:1, 11, or 13) encoding the “DEC2” proteins, for example, the polymerase chain reaction (PCR) method, can be utilized.

A protein functionally equivalent to a “DEC2” protein encoded by the DNA isolated through the above hybridization technique or gene amplification techniques normally has a high homology to the amino acid sequence of a “DEC2” protein (SEQ ID NO:2, 12, or 14). The proteins of the present invention also include proteins that are functionally equivalent to a “DEC2” protein and are highly homologous to the amino acid sequence shown in SEQ ID NO:2, 12, or 14. “Highly homologous” refers to, normally an identity of 60% or higher, preferably 70% or higher, more preferably 80% or higher. The homology of a protein can be determined by following the algorithm in “Wilbur, W. J. and Lipman, D. J. (1983) Proc. Natl. Acad. Sci. USA 80:726-730”.

The proteins of the present invention may have variations in the amino acid sequence, molecular weight, isoelectric point, the presence or absence of sugar chains, form, and so on, depending on the cell or host used to produce it or the purification method utilized (described below). Nevertheless, as long as the obtained protein has a function equivalent to a “DEC2” protein, it is within the scope of the present invention. For example, if a protein of the present invention is expressed in a prokaryotic cell such as E. coli, the protein includes a methionine residue at the N-terminus in addition to the natural amino acid sequence of the protein. Such proteins are also included in the proteins of the present invention.

The proteins of the present invention can be prepared recombinant proteins or naturally occurring proteins, using methods commonly known in the art. When the protein is a recombinant protein, it may be produced by inserting a DNA (for example, a DNA having the nucleotide sequence of SEQ ID NO:1, 11, or 13) encoding a protein of the present invention into an appropriate expression vector, collecting the transformant obtained by introducing the vector into an appropriate host cell, obtaining an extract, and then purifying and preparing the protein using ion exchange, reverse phase, gel filtration, or affinity chromatography. Affinity chromatography may be done using a column in which an antibody against a protein of the present invention is fixed. A combination of such columns may also be used.

Alternatively, when a protein of the invention is expressed in host cells (e.g., animal cells or E. coli) as a fusion protein with glutathione S transferase protein, or a recombinant protein with multiple histidine residues, the expressed recombinant protein can be purified using a glutathione column or nickel column.

After the fusion protein is purified, if necessary, regions of the fusion protein (apart from the desired protein) can be digested and removed with thrombin, factor Xa, etc.

The native protein of the invention can be isolated by methods well known in the art, for example, purifying an extract of tissues or cells that express a protein of the invention with an affinity column to which an antibody binding to a protein of the present invention described below is bound. The antibody may be a polyclonal or monoclonal antibody.

The term “substantially pure” as used herein in reference to a given polypeptide means that the polypeptide is substantially free from other biological macromolecules. For example, the substantially pure polypeptide is at least 75%, 80, 85, 95, or 99% pure by dry weight. Purity can be measured by any appropriate standard method known in the art, for example, by column chromatography, polyacrylamide gel electrophoresis, or HPLC analysis.

Accordingly, the invention includes a polypeptide having a sequence shown as SEQ ID NO:2, SEQ ID NO:12, or SEQ ID NO:14. The invention also includes a polypeptide, or fragment thereof, that differs from the corresponding sequence shown as SEQ ID NO:2, SEQ ID NO:12, or SEQ ID NO:14. The differences are, preferably, differences or changes at a non-essential residue or a conservative substitution. In one embodiment, the polypeptide includes an amino acid sequence at least about 60% identical to a sequence shown as SEQ ID NO:2, SEQ ID NO:12, SEQ ID NO:14, or a fragment thereof Preferably, the polypeptide is at least 65%, 70%, 75%, 80%, 85%, 90%, 95%, 98%, 99% or more identical to SEQ ID NO:2, SEQ ID NO:12, or SEQ ID NO:14 and has at least one transcriptional regulation function or activity described herein. Preferably, the polypeptide has binding activity towards CANNTG and/or CACNAG. Preferred polypeptide fragments of the invention are at least 10%, preferably at least 20%, 30%, 40%, 50%, 60%, 70%, or more, of the length of the sequence shown as SEQ ID NO:2, SEQ ID NO:12, or SEQ ID NO:14 and have at least one transcriptional regulation function or activity described herein. Or alternatively, the fragment can be merely an immunogenic fragment.

The present invention also includes partial peptides of the proteins of the present invention. The partial peptides of the present invention comprise at least 8 or more amino acids, preferably 15 or more amino acids, more preferably 30 or more amino acids, and still more preferably 50 or more amino acids (for example, 100 amino acids or more). The partial peptides can be used, for example, for generating antibodies against a protein of the present invention, screening of compounds binding to a protein of the present invention, or screening of stimulators or inhibitors of a protein of the present invention. Additionally, they may be antagonists or competitive inhibitors of the proteins of the present invention. The partial peptides of the proteins of the present invention include those that include, for example, the bHLH region of a protein comprising the amino acid sequence shown in SEQ ID NO:2, 12, or 14.

The partial peptides of the present invention can be produced by genetic engineering methods, known peptide synthesis methods, or by cutting the proteins of the present invention by appropriate peptidases. Synthesis of the peptides may be conducted according to, for example, the solid phase synthesis method, or the liquid phase synthesis method.

In addition to being utilized in the above-described in vivo or in vitro production of a protein of the present invention, a DNA encoding a protein of the present invention may also be applied, for example, in the gene therapy of diseases caused by an aberration in a gene encoding a protein of the present invention or diseases treatable by a protein of the present invention. Any type of DNA, such as cDNA synthesized from mRNA, genomic DNA, or synthetic DNA can be used so long as the DNA encodes a protein of the present invention. Also as long as they can encode a protein of the present invention, DNA comprising arbitrary sequences based on the degeneracy of the genetic code are also included.

The DNA of the present invention can be prepared by using methods known in the art. For example, a cDNA library can be constructed from cells expressing a protein of the present invention and hybridization can be conducted using a part of the DNA sequence of the present invention (for example, SEQ ID NO:1, 11, or 13) as a probe. The cDNA library may be prepared, for example, according to the method described by Sambrook et al. (Molecular Cloning, Cold Spring Harbor Laboratory Press, 1989), or instead, commercially available cDNA libraries may be used. Alternatively, a DNA of the present invention can be obtained by preparing RNA from cells expressing a protein of the present invention, synthesizing cDNA by using a reverse transcriptase, synthesizing oligo-DNA based on a DNA sequence of the present invention (for example, SEQ ID NO:1, 11, or 13), and amplifying the cDNA encoding a protein of the present invention by PCR using the oligo-DNA as primers.

The nucleotide sequence of the obtained cDNA is determined to find an open reading frame, and thereby, the amino acid sequence of a protein of the invention can be obtained. The cDNA obtained may also be used as a probe for screening a genomic library to isolate genomic DNA.

More specifically, mRNA may first be isolated from a cell, tissue, or organ in which a protein of the invention is expressed (e.g., tissues such as brain, skeletal muscle, testis, placenta, large intestine, spleen, and cartilage). Known methods can be used to isolate mRNA; for instance, total RNA is prepared by guanidine ultracentrifugation (Chirgwin et al., Biochemistry 18:5294-5299, 1979) or AGPC method (Chomczynski et al., Anal. Biochem. 162:156-159, 1987), and mRNA is purified from total RNA using an mRNA Purification Kit (Pharmacia) and such. Alternatively, mRNA may be directly prepared by QuickPrep mRNA Purification Kit (Pharmacia).

The obtained mRNA is used to synthesize cDNA using reverse transcriptase. cDNA may be synthesized by using a kit such as the AMV Reverse Transcriptase First-strand cDNA Synthesis Kit (Seikagaku Kogyo). Alternatively, cDNA may be synthesized and amplified following the 5′-RACE method (Frohman et al., Proc. Natl. Acad. Sci. USA 85:8998-9002, 1988; Belyavsky et al., Nucleic Acids Res. 17:2919-2932, 1989) that uses primers and such described herein, the 5′-Ampli FINDER RACE Kit (Clontech), and polymerase chain reaction (PCR).

A desired DNA fragment is prepared from the PCR products and linked to a vector DNA. The recombinant vector is used to transform E. coli and such, and the desired recombinant vector is prepared from a selected colony. The nucleotide sequence of the desired DNA can be verified by conventional methods, such as dideoxynucleotide chain termination.

A DNA of the invention may be designed to have a sequence that is expressed more efficiently by taking into account the frequency of codon usage in the host used for expression (Grantham et al., Nucleic Acids Res. 9:43-74, 1981). The DNA of the present invention may be altered by a commercially available kit or a conventional method. For instance, the DNA may be altered by digestion with restriction enzymes, insertion of a synthetic oligonucleotide or an appropriate DNA fragment, addition of a linker, or insertion of the initiation codon (ATG) and/or the stop codon (TAA, TGA, or TAG), etc.

Specifically, the DNA of the present invention include DNA having the following nucleotide sequences: from A at position 135 to C at position 1580 of SEQ ID NO:1, from A at position 2 to C at position 1453 of SEQ ID NO:11, and from A at position 74 to C at position 1303 of SEQ ID NO:13.

Furthermore, the DNA of the present invention include DNA capable of hybridizing with DNA having the nucleotide sequence of SEQ ID NO:1, 11, or 13 under stringent conditions, and encoding a protein functionally equivalent to a protein of the invention described above. Stringent hybridization conditions may be appropriately chosen by one skilled in the art. Specifically, conditions described above may be used. Under the conditions, DNA having higher homologies can be obtained by increasing temperature. The above hybridizing DNA is preferably a natural DNA, for example, a cDNA or a chromosomal DNA.

As used herein, an “isolated nucleic acid” is a nucleic acid, the structure of which is not identical to that of any naturally occurring nucleic acid or to that of any fragment of a naturally occurring genomic nucleic acid spanning more than three genes. The term therefore covers, for example, (a) a DNA which has the sequence of part of a naturally occurring genomic DNA molecule but is not flanked by both of the coding sequences that flank that part of the molecule in the genome of the organism in which it naturally occurs; (b) a nucleic acid incorporated into a vector or into the genomic DNA of a prokaryote or eukaryote in a manner such that the resulting molecule is not identical to any naturally occurring vector or genomic DNA; (c) a separate molecule such as a cDNA, a genomic fragment, a fragment produced by polymerase chain reaction (PCR), or a restriction fragment; and (d) a recombinant nucleotide sequence that is part of a hybrid gene, i.e., a gene encoding a fusion protein. Specifically excluded from this definition are nucleic acids present in random, uncharacterized mixtures of different DNA molecules, transfected cells, or cell clones, e.g., as these occur in a DNA library such as a cDNA or genomic DNA library.

Accordingly, in one aspect, the invention provides an isolated or purified nucleic acid molecule that encodes a polypeptide described herein or a fragment thereof. Preferably, the isolated nucleic acid molecule includes a nucleotide sequence that is at least 60% identical to the nucleotide sequence shown in SEQ ID NO:1, SEQ ID NO:11, or SEQ ID NO:13. More preferably, the isolated nucleic acid molecule is at least 65%, 70%, 75%, 80%, 85%, 90%, 91%, 92%, 93%, 94%, 95%, 96%, 97%, 98%, 99%, or more, identical to the nucleotide sequence shown in SEQ ID NO:1, SEQ ID NO:11, or SEQ ID NO:13. In the case of an isolated nucleic acid molecule which is longer than or equivalent in length to the reference sequence, e.g., SEQ ID NO:1, SEQ ID NO:1 1, or SEQ ID NO:13, the comparison is made with the full length of the reference sequence. Where the isolated nucleic acid molecule is shorter that the reference sequence, e.g., shorter than SEQ ID NO:1, SEQ ID NO:11, or SEQ ID NO:13, the comparison is made to a segment of the reference sequence of the same length (excluding any loop required by the homology calculation).

As used herein, “% identity” of two amino acid sequences, or of two nucleic acid sequences, is determined using the algorithm of Karlin and Altschul (PNAS USA 87:2264-2268, 1990), modified as in Karlin and Altschul, PNAS USA 90:5873-5877, 1993). Such an algorithm is incorporated into the NBLAST and XBLAST programs of Altschul et al. (J. Mol. Biol. 215:403-410, 1990). BLAST nucleotide searches are performed with the NBLAST program, score=100, wordlength=12. BLAST protein searches are performed with the XBLAST program, score=50, wordlength=3. To obtain gapped alignment for comparison purposes GappedBLAST is utilized as described in Altschul et al. (Nucleic Acids Res. 25:3389-3402, 1997). When utilizing BLAST and GappedBLAST programs the default parameters of the respective programs (e.g., XBLAST and NBLAST) are used to obtain nucleotide sequences homologous to a nucleic acid molecule of the invention.

The present invention also provides a vector into which a DNA of the present invention is inserted. The vectors of the present invention are useful in maintaining the DNA of the present invention within the host cell, or expressing a protein of the present invention.

When E. coli is used as the host cell, there is no limitation other than that the vector should have an “ori”, to amplify and mass-produce the vector in E. coli (e.g., JM109, DH5α, HB 101, or XL1Blue), and such, and a marker gene for selecting the transformed E. coli (e.g., a drug-resistance gene selected by a drug (e.g., ampicillin, tetracycline, kanamycin, or chloramphenicol)). For example, M13-series vectors, pUC-series vectors, pBR322, pBluescript, pCR-Script, and such can be used. Besides the vectors, pGEM-T, pDIRECT, pT7, and so on can also be used for the subcloning and excision of the cDNA as well. When a vector is used to produce a protein of the present invention, an expression vector is especially useful. When the expression vector is expressed, for example, in E. coli, it should have the above characteristics in order to be amplified in E. coli. Additionally, when E. coli, such as JM109, DH5α, HB101, or XL1-Blue, are used as the host cell, the vector should have a promoter, e.g., lacZ promoter (Ward et al., Nature 341:544-546, 1989; FASEB J. 6:2422-2427, 1992), araB promoter (Better et al., Science 240:1041-1043, 1988), or T7 promoter, that can efficiently promote the expression of the desired gene in E. coli. Other examples of the vectors are pGEX-5X-1 (Pharmacia), “QIAexpress system” (QIAGEN), pEGFP, and pET (for this vector, BL21, a strain expressing T7 RNA polymerase, is preferably used as the host).

Further, the vector may comprise a signal sequence to secrete the polypeptide. For producing the protein into the periplasm of E. coli, the pelB signal sequence (Lei et al., J. Bacteriol. 169:4379, 1987) may be used as the signal sequence for protein secretion. For example, the calcium chloride method or electroporation may be used to introduce the vector into host cells.

As vectors used to produce the proteins of the present invention, for example, expression vectors derived from mammals (e.g., pCDNA3 (Invitrogen), pEGF-BOS (Nucleic Acids Res. 18(17):5322, 1990), pEF, pCDM8), insect cells (e.g., “Bac-to-BAC baculovirus expression system” (GIBCO-BRL), pBacPAK8), plants (e.g., pMH1, pMH2), animal viruses (e.g., pHSV, pMV, pAdexLcw), retroviruses (e.g., ZIPneo), yeasts (e.g., “Pichia Expression Kit” (Invitrogen), pNV11, SP-Q01), and Bacillus subtilis (e.g., pPL608, pKTH50) can be mentioned other than E. coli.

In order to express proteins in animal cells, such as CHO, COS, and NIH3T3 cells, the vector must have a promoter necessary for expression in such cells (e.g., SV40 promoter (Mulligan et al., Nature 277:108, 1979), MMLV-LTR promoter, EF1α promoter (Mizushima et al., Nucleic Acids Res. 18:5322, 1990), CMV promoter, etc.). It is more preferable if the vector additionally had a marker gene for selecting transformants (for example, a drug resistance gene selected by a drug (e.g., neomycin, G418, etc.)). Examples of vectors with such characteristics include pMAM, pDR2, pBK-RSV, pBK-CMV, pOPRSV, pOP 13, and so on.

Furthermore, in order to stably express the gene and to amplify the copy number in cells, the method using CHO cells deficient in nucleic acid synthetic pathways as the host, incorporating into the CHO cells a vector (such as pCHOI) having a DHFR gene that compensates for the deficiency, and amplifying the vector with methotrexate (MTX) can be used. Furthermore, for transiently expressing a gene, the method that transforms COS cells that have the gene for SV40 T antigen on the chromosome with a vector (such as pcD) having the SV40 replication origin can be mentioned. The replication origin may be that of a polyomavirus, adenovirus, bovine papilloma virus (BPV), and the like. Further, to amplify the gene copy number in the host cells, selection markers such as the aminoglycoside transferase (APH) gene, thymidine kinase (TK) gene, E. coli xanthine-guanine phosphoribosyl transferase (Ecogpt) gene, and the dihydrofolate reductase (dhfr) gene may be comprised in the expression vector.

A DNA of the present invention can be expressed in animals by, for example, inserting a DNA of the invention into an appropriate vector and introducing the vector into a living body by the retrovirus method, liposome method, cationic liposome method, adenovirus method, and so on. Thus, it is possible to perform gene therapy of diseases caused by a mutation of the “DEC2” gene of the present invention. The vectors used in these methods include, but are not limited to, adenovirus vectors (e.g., pAdexlcw), retrovirus vectors (e.g., pZIPneo), and so on. General techniques for gene manipulation, such as insertion of the DNA of the invention into a vector, can be performed according to conventional methods (Molecular Cloning, 5.61-5.63). Administration to the living body may be performed according the ex vivo method or the in vivo method.

The present invention also provides a host cell into which a vector of the present invention has been introduced. The host cell into which the vector of the invention is introduced is not particularly limited. For example, E. coli, various animal cells, and such, can be used. The host cell of the present invention can be used, for example, as a production system to produce and express a protein of the present invention. Protein production systems include in vitro and in vivo systems. Such production systems using eukaryotic cells or prokaryotic cells can be given as in vitro production systems.

As eukaryotic host cells, for example, animal cells, plant cells, and fungi cells can be used. Mammalian cells, for example, CHO (J. Exp. Med. 108:945, 1995), COS, 3T3, myeloma, BHK (baby hamster kidney), HeLa, Vero, amphibian cells (e.g., platanna oocytes (Valle et al., Nature 291:358-340, 1981), and insect cells (e.g., Sf9, Sf21, Tn5) are known as animal cells. Among CHO cells, those deficient in the DHFR gene, dhfr-CHO (Proc. Natl. Acad. Sci. USA 77:4216-4220, 1980) and CHO K-1 (Proc. Natl. Acad. Sci. USA 60:1275, 1968), are particularly preferable. Among animal cells, CHO cells are particularly preferable for mass expression. A vector can be introduced into a host cell by, for example, the calcium phosphate method, the DEAE-dextran method, methods using cationic liposome DOTAP (Boehringer-Mannheim), electroporation, lipofection, etc.

As plant cells, for example, plant cells originating from Nicotiana tabacum are known as protein producing systems and may be used as callus cultures. As fungal cells, yeast cells such as Saccharomyces, including Saccharomyces cerevisiae, or filamentous fungi such as Aspergillus, including Aspergillus niger, are known.

Useful prokaryotic cells include bacterial cells. Bacterial cells such as E. coli, for example, JM109, DH5α, HB101, and such, as well as Bacillus subtilis are known.

These cells are transformed by a desired DNA, and the resulting transformants are cultured in vitro to obtain the protein. Transformants can be cultured using known methods. For example, culture medium such as DMEM, MEM, RPMI1640, or IMDM may be used with or without serum supplements such as fetal calf serum (FCS) as culture medium for animal cells. The pH of the culture medium is preferably between about 6 and 8. Such cells are typically cultured at about 30 to 40° C. for about 15 to 200 hr, and the culture medium may be replaced, aerated, or stirred if necessary.

Animal and plant hosts may be used for in vivo production. For example, a desired DNA can be introduced into an animal or plant host. Encoded proteins are produced in vivo, and then recovered. These animal and plant hosts are included in the “host” of the present invention.

Animals to be used for the production system described above include mammals and insects. Mammals such as goats, pigs, sheep, mice, and cattle may be used (Vicki Glaser, SPECTRUM Biotechnology Applications, 1993). Alternatively, the mammals may be transgenic animals.

For instance, a desired DNA may be prepared as a fusion gene with a gene such as goat β casein gene that encodes a protein specifically produced into milk. DNA fragments comprising the fusion gene are injected into goat embryos, which are then introduced back to female goats. Proteins are recovered from milk produced by the transgenic goats (i.e., those born from the goats that had received the modified embryos) or from their offspring. To increase the amount of milk containing the proteins produced by transgenic goats, appropriate hormones may be administered (Ebert et al., Bio/Technology 12:699-702, 1994).

Alternatively, insects, such as the silkworm, may be used. Baculoviruses into which a DNA encoding a desired protein has been inserted can be used to infect silkworms, and the desired protein is recovered from the body fluid (Susumu et al., Nature 315:592-594, 1985).

As plants, for example, tobacco can be used. When using tobacco, a DNA encoding a desired protein may be inserted into a plant expression vector, such as pMON 530, which is introduced into bacteria, such as Agrobacterium tumefaciens. Then, the bacteria is used to infect tobacco, such as Nicotiana tabacum, and the desired polypeptide is recovered from the leaves (Julian et al., Eur. J. Immunol. 24:131-138, 1994).

A protein of the present invention obtained as above may be isolated from inside or outside of hosts (medium, etc.), and purified as a substantially pure homogeneous protein. The method for protein isolation and purification is not limited to any specific method; in fact, any standard method may be used. For instance, column chromatography, filters, ultrafiltration, salting out, solvent precipitation, solvent extraction, distillation, immunoprecipitation, SDS-polyacrylamide gel electrophoresis, isoelectric point electrophoresis, dialysis, and recrystallization may be appropriately selected and combined to isolate and purify the protein.

For chromatography, for example, affinity chromatography, ion-exchange chromatography, hydrophobic chromatography, gel filtration chromatography, reverse phase chromatography, adsorption chromatography, and such may be used (Strategies for Protein Purification and Characterization: A Laboratory Course Manual. Ed., Daniel R. Marshak et al., Cold Spring Harbor Laboratory Press, 1996). These chromatographies may be performed by liquid chromatographies such as HPLC and FPLC. Thus, the present invention provides highly purified proteins produced by the above methods.

A protein may be optionally modified or partially deleted by treating it with an appropriate protein-modifying enzyme before or after purification. For example, trypsin, chymotrypsin, lysylendopeptidase, protein kinase, glucosidase, and such are used as protein-modifying enzymes.

The present invention also provides antibodies binding to a protein of the present invention. The antibodies of the present invention may take any form, including monoclonal antibodies, as well as polyclonal antibodies. Furthermore, antiserum obtained by immunizing animals such as rabbits and the like with a protein of the invention, all classes of polyclonal and monoclonal antibodies, as well as human and humanized antibodies produced by genetic recombination are included.

A protein of the invention used as a sensitizing antigen to obtain antibodies may be derived from any animal species, but preferably it is from a mammal such as human, mouse, or rat, and more preferably from a human. A human-derived protein may be obtained by using a nucleotide or amino acid sequence disclosed herein.

A full-length protein or a partial peptide thereof may be used as a sensitized antigen in the present invention. A partial peptide may be, for example, an amino (N)-terminus or carboxy (C)-terminus fragment of the protein. Herein, an “antibody” is defined as an antibody that reacts with either the full length or a fragment of the protein.

A gene encoding a protein of the invention or its fragment may be inserted into a known expression vector used to transform a host cell as described herein. The desired protein or its fragment may be recovered from the outside or inside of host cells by any standard method, and may be used as the sensitized antigen. Alternatively, cells expressing the protein or their lysates, or a chemically synthesized protein may be used as an antigen. Preferably, short peptides are used as antigens by appropriately binding to carrier proteins such as keyhole limpet hemocyanin, bovine serum albumin, and ovalbumin.

Any mammal may be immunized with the sensitized antigen, but preferably, the compatibility with parental cells used for cell fusion is taken into account. In general, animals of Rodentia, Lagomorpha, or Primates are used.

Animals of Rodentia include, for example, mice, rats, and hamsters. Animals of Lagomorpha include, for example, rabbits. Animals of Primates include, for example, monkeys of Catarrhini (old world monkeys) such as Macaca fascicularis, rhesus monkeys, sacred baboons, or chimpanzees.

Methods for immunizing animals with antigens are well known. Intraperitoneal injection or subcutaneous injection of antigens is used as a standard method. More specifically, antigens may be diluted and suspended in an appropriate amount with phosphate buffered saline (PBS), physiological saline, etc. If desired, the antigen suspension may be mixed with an appropriate amount of a standard adjuvant, such as Freund's complete adjuvant, made into an emulsion, and then administered to mammals. Preferably, this is followed by several administrations of the antigen mixed with an appropriate amount of Freund's incomplete adjuvant every 4 to 21 days. An appropriate carrier may also be used for immunization. After the above immunization, the serum is examined for an increase of the amount of desired antibodies by a standard method.

Polyclonal antibodies raised against a protein of the present invention may be prepared by collecting blood from the immunized mammal after confirming the increase of desired antibodies in the serum, and by separating serum from the blood by any conventional method. Serum containing a polyclonal antibody may also be used as a polyclonal antibody, or if necessary, the fraction containing the polyclonal antibody may be isolated from the serum. For example, fractions that recognize only a protein of the present invention are obtained by using affinity columns to which the present protein is coupled, and by further purifying the fraction using a protein A or G column, immunoglobulin G or M may be prepared.

To prepare monoclonal antibodies, immune cells are collected from a mammal immunized with an antigen and checked for an increase of the level of the desired antibodies in the serum as described above, and these cells are subjected to cell fusion. The immune cells used for cell fusion are preferably obtained from the spleen. The other parent cell fused with the above immune cell is preferably a mammalian myeloma cell, and more preferably, a myeloma cell that has acquired a special feature that can be used for selecting fusion cells by a drug.

The above immune cell and myeloma cell may be fused by basically any standard method, such as those described in literature (Galfre et al., Methods Enzymol. 73:3-46, 1981).

Resulting hybridomas obtained by the cell fusion may be selected by cultivating them in a standard selection medium, such as the HAT medium (hypoxanthine, aminopterin, and thymidine containing medium). The cell culture is typically continued in the HAT medium for a period of time that is sufficient to allow all cells except the desired hybridoma (non-fused cells) to die, usually from several days to several weeks. Then, standard limiting dilution is performed to screen and clone a hybridoma cell producing the desired antibody.

Besides the above method in which a nonhuman animal is immunized with an antigen for preparing a hybridoma, human lymphocytes such as those infected by the EB virus may be immunized with a protein, protein-expressing cells, or their lysates in vitro. Then, the immunized lymphocytes are fused with human-derived myeloma cells that are capable of indefinite division, such as U266, to yield a hybridoma producing a desired human antibody capable of binding to a protein of the invention (JP-A No. Sho 63-17688).

Subsequently, the hybridomas thus obtained are transplanted into the abdominal cavity of a mouse from which the ascites is collected. The monoclonal antibodies thus obtained can be purified by, for example, ammonium sulfate precipitation or column chromatography using a protein A or protein G column, a DEAE ion exchange column, an affinity column and such to which a protein of the invention is coupled. An antibody of the invention can be used not only for purifying and detecting a protein of the invention, but also as a candidate for an agonist or antagonist of a protein of the present invention. Such an antibody is also expected to be used for antibody therapy of diseases in which the proteins of this invention are involved. To administer the obtained antibody to human bodies (namely, antibody therapy), human antibodies or humanized antibodies are preferred to reduce immunogenicity.

For example, transgenic animals having a repertory of human antibody genes may be immunized with a protein, protein expressing cells, or their lysates as antigen. Antibody producing cells are collected from the animals, and fused with myeloma cells to obtain hybridoma, from which human antibodies against the protein can be prepared (see WO92-03918, WO93-2227, WO94-02602, WO94-25585, WO96-33735, and WO96-34096).

Alternatively, an immune cell that produces antibodies, such as an immunized lymphocyte, may be immortalized by an oncogene and used for preparing monoclonal antibodies.

Such monoclonal antibodies can also be recombinantly prepared using genetic engineering techniques (see, for example, Borrebaeck C. A. K. and Larrick J. W. Therapeutic Monoclonal Antibodies, published in the United Kingdom by MacMillan Publishers LTD, 1990). A recombinant antibody can be prepared by cloning a DNA encoding the antibody from an immune cell such as a hybridoma or an immunized lymphocyte producing the antibody, inserting this into an appropriate vector, and introducing the vector into an host cell. The present invention also encompasses recombinant antibodies prepared as described above.

An antibody of the present invention may be a fragment of an antibody or modified antibody, so long as it binds to one or more of the proteins of the invention. For instance, the antibody fragment may be Fab, F(ab′)₂, Fv, or single chain Fv (scFv), in which Fv fragments from H and L chains are linked by an appropriate linker (Huston et al., Proc. Natl. Acad. Sci. USA 85:5879-5883, 1988). More specifically, an antibody fragment may be generated by treating an antibody with an enzyme such as papain or pepsin. Alternatively, a gene encoding the antibody fragment may be constructed, inserted into an expression vector, and expressed in an appropriate host cell (see, for example, Co et al., J. Immunol. 152:2968-2976, 1994; Better et al., Methods Enzymol. 178:476-496, 1989; Pluckthun et al., Methods Enzymol. 178:497-515, 1989; Lamoyi, Methods Enzymol. 121:652-663, 1986; Rousseaux et al., Methods Enzymol. 121:663-669, 1986; Bird et al., Trends Biotechnol. 9:132-137, 1991).

An antibody may be modified by conjugation with a variety of molecules, such as polyethylene glycol (PEG). The present invention provides such modified antibodies. A modified antibody can be obtained by chemically modifying an antibody. These modification methods are conventional in the field.

Alternatively, an antibody of the present invention may be obtained as a chimeric antibody comprising a variable region derived from a nonhuman antibody and the constant region derived from a human antibody, or as a humanized antibody comprising the complementarity determining region (CDR) derived from a nonhuman antibody, the framework region (FR) derived from a human antibody, and the constant region, by using well-known methods.

Obtained antibodies may be purified to homogeneity. Any standard method protein separation and purification method may be used for antibody separation and purification. For example, chromatographies such as affinity chromatography, filters, ultrafiltration, salting out, dialysis, SDS polyacrylamide gel electrophoresis, isoelectric point electrophoresis, and such may be appropriately combined to isolate and purify the antibody (Antibodies: A Laboratory Manual. Ed Harlow and David Lane, Cold Spring Harbor Laboratory, 1988). However, the methods are not limited thereto. The concentration of the obtained antibody may be determined by measuring absorbance, by enzyme-linked immunosorbent assay (ELISA), etc.

Columns used for affinity chromatography include, protein A column and protein G column. For example, Hyper D, POROS, Sepharose F.F. (Pharmacia), and such may be mentioned as columns using protein A columns.

Chromatographies other than affinity chromatography are, for example, ion exchange chromatography, hydrophobic chromatography, gel filtration chromatography, reverse phase chromatography, adsorption chromatography, and so on (Strategies for Protein Purification and Characterization: A Laboratory Course Manual. Ed Daniel R. Marshak et al., Cold Spring Harbor Laboratory Press, 1996). These chromatographies can be conducted using liquid chromatographies such as HPLC, and FPLC.

For example, measurement of absorbance, enzyme-linked immunosorbent assay (ELISA), enzyme immunoassay (EIA), radioimmunoassay (RIA), or immunofluorescence may be used to measure the antigen binding activity of an antibody of the invention. In ELISA, an antibody of the present invention is immobilized on a plate, a protein of the invention is applied, and then a sample containing a desired antibody, such as culture supernatant of antibody producing cells or a purified antibody, is applied. Then, a secondary antibody labeled with an enzyme such as alkaline phosphatase that recognizes the primary antibody is applied, and the plate is incubated. After washing, an enzyme substrate, such as p-nitrophenyl phosphate, is added to the plate, and the absorbance is measured to evaluate the antigen binding activity of the sample. A fragment of a protein, such as the C-terminus fragment, may be used as the protein. BIAcore (Pharmacia) may be used to evaluate the activity of an antibody according to the present invention.

The above methods allow the detection or measurement of a protein of the invention, by exposing an antibody of the invention to a sample assumed to contain the protein of the invention, and detecting or measuring the immune complex formed by the antibody and the protein. Because the method of detection or measurement of a protein according to the invention can specifically detect or measure a protein, the method may be useful in a variety of experiments in which the protein is used.

The present invention also provides DNA comprising at least 15 nucleotides that is complementary to a DNA encoding a “DEC2” protein (SEQ ID NO:1, 11, or 13) or to the complementary strand thereof.

“Complementary strand” herein refers to one strand of a double strand DNA comprising A:T and G:C base pairs, when viewed against the other strand. Furthermore, “complementary” means not only when a nucleotide sequence is completely complementary to a continuous nucleotide sequence with at least 15 nucleotides, but also when there is a homology of at least 70%, preferably at least 80%, more preferably 90%, and much more preferably 95% or more at the nucleotide sequence level. Homology can be determined by using the algorithm described herein.

Such DNA include probes and primers used for the detection and amplification of a DNA encoding a protein of the present invention, nucleotides and nucleotide derivatives (for example, antisense oligonucleotides or DNA encoding ribozymes, etc.) used for repressing the expression of a protein of the present invention. Furthermore, such DNA can be used in the preparation of DNA chips.

If the DNA is used as a primer, the 3′ region thereof may be the complementary site, and restriction enzyme recognition sites, tag sequences, and such may be attached to the 5′ region.

Antisense oligonucleotides comprise, for example, an antisense oligonucleotide that hybridizes with any portion of the nucleotide sequence of SEQ ID NO:1, 11, or 13. The antisense oligonucleotide is preferably an antisense of a continuous sequence comprising at least 15 nucleotides or more within the nucleotide sequence of SEQ ID NO:1, 11, or 13. More preferably, the above continuous sequence comprising at least 15 nucleotides or more contains a translation initiation codon.

A derivative or modified form of an antisense oligonucleotide may also be used. The latter form may be prepared by modifying an antisense oligonucleotide with lower alkylphosphonates such as methylphosphonate or ethylphosphonate, or with phosphorothioate, or phosphoroamidate.

The antisense oligonucleotide is not restricted to one in which all nucleotides are complementary to the corresponding nucleotides within a given region of a DNA or mRNA. As long as it can specifically hybridize with the nucleotide sequences of SEQ ID NO:1, 11, or 13, it may have one or more nucleotide mismatches.

A derivative of an antisense oligonucleotide of the present invention may act on cells producing a protein of the invention and bind to a DNA or mRNA encoding the protein, and then, it may inhibit the expression of the protein of the invention by inhibiting its transcription or translation, or by promoting the degradation of mRNA, and thereby inhibiting the function of the protein.

A derivative of an antisense oligonucleotide of the present invention may be mixed with an appropriate base that is inactive against the derivative, and used as a medicine for external application, such as a salve or poultice.

If necessary, it may be mixed with an excipient, isotonizing agent, solubilizing agent, stabilizer, preservative, pain-killer, or the like, and prepared as a tablet, powder, granule, capsule, liposome capsule, injectable solution, liquid formulation, nose drops, freeze-dried agent, etc. The above may be achieved according to standard methods.

For treating patients, a derivative of an antisense oligonucleotide of the present invention may be, for example, directly applied to the affected area of a patient, or administered into blood vessels so as to finally reach the affected area. Moreover, the derivative may be encapsulated in antisense-encapsulating materials such as liposomes, poly-L-lysine, lipid, cholesterol, lipofectin, or their derivatives in order to increase durability and/or membrane permeability.

Dose of the derivative of the antisense oligonucleotide of the present invention may be appropriately adjusted depending on the patient's conditions, and a favorable amount such as 0.1 to 100 mg/kg, or more, preferably 0.1 to 50 mg/kg, may be administered.

As an antisense oligonucleotide of the present invention inhibits the expression of a protein of the invention, it is useful as an inhibitor of a biological activity of the protein of the invention. An inhibitor of expression comprising an antisense oligonucleotide of the present invention is useful due to its ability to inhibit a biological activity of a protein of the invention.

A protein of the invention may be useful for screening a compound that binds to the protein. Specifically, the protein may be used in a method of screening for the compound, such a method comprising the steps of exposing the protein of the present invention to a test sample expected to contain a compound binding to the protein, and selecting a compound having the activity of binding to the protein.

Proteins of the invention used for screening may be recombinant or natural proteins, or partial peptides. Alternatively, they may be in the form expressed on the surface of a cell, or in the form of a membrane fraction. Samples tested include, but are not limited to, cell extracts, cell culture supernatants, products of fermentation microorganisms, marine organism extracts, plant extracts, purified or crude preparations of proteins, peptides, non-peptide compounds, synthetic low-molecular weight compounds, and natural compounds. A protein of the present invention contacted with a test sample may be brought into contact with the test sample, for example, as a purified protein, as a soluble protein, in the form attached to a carrier, a fusion protein with other proteins, in the form expressed on the cell membrane, or as a membrane fraction.

Various methods known to one skilled in the art may be used as the screening method of, for example, a protein that binds to a protein of the present invention (e.g., other bHLH type transcription factors and other proteins related to transcriptional regulation) using a protein of the present invention. Such a screening can be carried out, for example, by the immunoprecipitation method. Specifically, the method can be carried out as follows. A gene encoding a protein of this invention is expressed by inserting the gene into vectors for foreign gene expression such as pSV2neo, pcDNA I, and pCD8, and expressing the gene in animal cells, etc. Any generally used promoter may be employed for the expression, including the SV40 early promoter (Rigby In Williamson (ed.), Genetic Engineering, Vol. 3. Academic Press, London, p.83-141, 1982), EF-1 α promoter (Kim et al., Gene 91:217-223, 1990), CAG promoter (Niwa et al., Gene 108:193-200, 1991), RSV LTR promoter (Cullen, Methods in Enzymol. 152:684-704, 1987), SR α promoter (Takebe et al., Mol. Cell. Biol. 8:466, 1988), CMV immediate early promoter (Seed et al., Proc. Natl. Acad. Sci. USA 84:3365-3369, 1987), SV40 late promoter (Gheysen et al., J. Mol. Appl. Genet. 1:385-394, 1982), Adenovirus late promoter (Kaufman et al., Mol. Cell. Biol. 9:946, 1989), HSV TK promoter, etc. Transfer of a foreign gene into animal cells for expression therein can be performed by any of the following methods, including the electroporation method (Chu et al., Nucl. Acid Res. 15:1311-1326, 1987), the calcium phosphate method (Chen et al., Cell. Biol. 7:2745-2752, 1987), the DEAE dextran method (Lopata et al., Nucl. Acids Res. 12:5707-5717, 1984; Sussman et al., Mol. Cell. Biol. 4:1642-1643, 1985), the lipofectin method (Derijard, Cell. 7:1025-1037, 1994; Lamb et al., Nature Genetics 5:22-30, 1993; Rabindran et al., Science 259:230-234, 1993), etc. A protein of this invention can be expressed as a fusion protein having a recognition site for a monoclonal antibody whose specificity has been established by introducing the recognition site (epitope) into the N- or C-terminal of a protein of this invention. For this purpose, a commercial epitope-antibody system can be utilized (Jikken Igaku, Experimental Medicine 13:85-90, 1995). Vectors that are capable of expressing fusion proteins with p-galactosidase, maltose-binding protein, glutathione S-transferase, green fluorescence protein (GFP), and such, via a multi-cloning site are commercially available.

To minimize the alteration in properties of a protein of this invention due to fusion protein formation, a method for preparing a fusion protein by introducing only a small epitope portion comprising several to ten amino acid residues has been reported. For example, the epitopes of polyhistidine (His-tag), influenza hemagglutinin (HA), human c-myc, FLAG, Vesicular stomatitis virus glycoprotein (VSV-GP), T7 gene 10 protein (T7-tag), human herpes simplex virus glycoprotein (HSV-tag), E-tag (epitope on the monoclonal phage), and such, and monoclonal antibodies to recognize these epitopes can be utilized as the epitope-antibody system for screening proteins binding to the protein of this invention (Jikken Igaku, Experimental Medicine 13:85-90, 1995).

In immunoprecipitation, immune complexes are formed by adding these antibodies to the cell lysate prepared using suitable surfactants. This immune complex comprises a protein of this invention, a protein capable of binding to the protein, and an antibody. The immunoprecipitation can also be performed using an antibody to a protein of this invention, besides antibodies to the above-described epitopes. An antibody to a protein of this invention can be prepared by, for example, inserting a gene encoding a protein of this invention into an appropriate expression vector of E. coli to express it in the bacterium, purifying the protein thus expressed, and immunizing rabbits, mice, rats, goats, chicken, and such, with the purified protein. The antibody can also be prepared by immunizing the above-described animals with partial peptides of a protein of this invention.

Immune complexes can be precipitated using, for example, Protein A Sepharose and Protein G Sepharose when the antibody is a murine IgG antibody. In addition, when the protein of this invention is prepared as a fusion protein with the epitope of, for example, GST, and such, the immune complex can be formed using a substance that specifically binds to these epitopes, such as glutathione-Sepharose 4B, and such, giving the same result as in the case where the antibody for the protein of this invention is used.

Immune precipitation, in general, may be carried out according to, or following the method described in literature (Harlow et al., Antibodies, pp.511-552, Cold Spring Harbor Laboratory publications, New York, 1988).

SDS-PAGE is generally used for the analysis of immunoprecipitated proteins. Bound proteins can be analyzed based on the molecular weights of proteins using a gel of an appropriate concentration. In this case, although proteins bound to the protein of this invention, are in general hardly detectable by the usual protein staining method, such as Coomassie staining and silver staining, the detection sensitivity can be improved by culturing cells in a medium containing radio isotope-labeled ³⁵S-methionine and ³⁵S-cysteine to label proteins inside the cells, and detecting the labeled proteins. Once the molecular weight of the protein is determined, the desired protein can be purified directly from SDS-polyacrylamide gel and sequenced.

Isolation of a protein that binds to a protein of the present invention using the protein may be carried out by, for example, using the West-Western blotting method (Skolnik et al., Cell 65:83-90, 1991). Specifically, a cDNA library is constructed from cells, tissues, or organs (for example, tissues or cultured cells from brain, skeletal muscle, testis, placenta, large intestine, spleen, cartilages, and so on) in which a protein binding to a protein of the present invention is expected to be expressed, by using phage vectors (λgt11, ZAP, etc.). Then, this is expressed on LB-agarose, transferred to a filter membrane, which is reacted with a purified labeled protein of the invention. The plaques expressing proteins that bind to the protein of the invention can be identified by detecting the label. The protein of the invention may be labeled by a method utilizing the binding between biotin and avidin, or a method utilizing an antibody that specifically binds to the protein of the present invention, or a peptide or polypeptide (e.g., GST and such) that is fused to the protein of the present invention. Methods using radioisotope or fluorescence and such may also be used.

Alternatively, in another embodiment of the method for screening of the present invention, a two-hybrid system utilizing cells may be used (Fields et al., Trends Genet. 10:286-292, 1994; Dalton et al., Cell 68:597-612, 1992; “MATCHMAKER Two-Hybrid System”, “Mammalian MATCHMAKER Two-Hybrid Assay Kit”, “MATCHMAKER One-Hybrid System” (products of Clontech); “HybriZAP Two-Hybrid Vector System” (Stratagene)). The two-hybrid system can be used as follows: (1) a protein of the present invention or a partial peptide thereof is fused to the SRF DNA binding region or GAL4 DNA binding region and expressed in yeast cells; (2) a cDNA library, which expresses proteins as fusion proteins with VP16 or GAL4 transcription activating regions, is prepared from cells expected to express proteins binding to the protein of the present invention; (3) the library is introduced to above mentioned yeast cells; and (4) library-derived cDNA are isolated from the positive clones detected (positive clones can be confirmed by activation of reporter genes due to the binding of the present protein and the binding protein expressed in the yeast cell). The protein encoded by the cDNA can be obtained by introducing the isolated cDNA into E. coli and expressing it. Thus, a protein binding to a present protein or genes thereof can be prepared. For example, in addition the HIS3 gene, Ade2 gene, LacZ gene, CAT gene, luciferase gene, PAI-1 (Plasminogen activator inhibitor type1) gene, and so on, can be mentioned as reporter genes used in the 2-hybrid system, but are not restricted thereto.

Alternatively, a compound binding to a protein of the present invention can be screened by affinity chromatography. For example, when the compound is a protein, a protein of the invention is immobilized on a carrier of an affinity column, and a test sample, in which a protein capable of binding to the protein of the invention is presumed to be expressed, is applied to the column. The test sample used herein may be a cell extract, cell lysate, etc. After loading the test sample, the column is washed, and a protein bound to the protein of the invention can be obtained.

The DNA encoding the protein may be obtained by analyzing the amino acid sequence of the obtained protein, synthesizing oligo DNA based on the sequence information, and screening a cDNA library using the DNA as the probe.

A biosensor utilizing the surface plasmon resonance phenomenon may be used as a means for detecting or measuring a compound bound to the present invention. When such a biosensor is used, the interaction between a protein of the invention and a test compound can be observed at real-time as a surface plasmon resonance signal, using only a minute amount of proteins without labeling (for example, BIAcore, Pharmacia). Therefore, it is possible to evaluate the binding between a protein of the invention and a test compound using a biosensor such as BIAcore.

In addition, methods for isolating not only proteins, but also compounds binding to the proteins of the invention (including agonists and antagonists) are known in the art. Such methods include, for example, the method of screening for a binding molecule by contacting synthesized compounds or natural substance banks, or random phage peptide display libraries with an immobilized protein of the invention, and the high-throughput screening method using the combinatorial chemistry technique (Wrighton et al., Science 273:458-64, 1996; Verdine, Nature 384:11-13, 1996; Hogan, Jr., Nature 384:17-9, 1996).

A compound that binds to a protein of the present invention may be a candidate for a drug that can promote or inhibit the activity of the protein, and thus may be applied to the treatment of diseases caused by an abnormal expression, function, and such of the protein. Such a drug may also be applied against diseases that may be treated by controlling the activity of a protein of the present invention. Compounds, which are obtained by the screening method of this invention and which have the activity to bind to a protein of this invention whose partial structure is modified by an addition, deletion and/or substitution, are also included in compounds that bind to the proteins of this invention.

When using a compound binding to a protein of this invention, or a protein of this invention as a pharmaceutical agent for humans and other mammals, such as mice, rats, guinea-pigs, rabbits, chicken, cats, dogs, sheep, pigs, cattle, monkeys, baboons, and chimpanzees, the protein or the isolated compound can be directly administered or can be formulated using known pharmaceutical preparation methods. For example, according to the need, the drugs can be taken orally as sugarcoated tablets, capsules, elixirs and microcapsules or non-orally in the form of injections of sterile solutions or suspensions with water or any other pharmaceutically acceptable liquid. For example, the compounds can be mixed with pharmacologically acceptable carriers or medium, specifically, sterilized water, physiological saline, plant-oil, emulsifiers, solvents, surfactants, stabilizers, flavoring agents, excipients, vehicles, preservatives and binders, in a unit dose form required for generally accepted drug implementation. The amount of active ingredients in these preparations makes a suitable dosage within the indicated range acquirable.

Examples of additives that can be mixed to tablets and capsules are, binders such as gelatin, corn starch, tragacanth gum and arabic gum; excipients such as crystalline cellulose; swelling agents such as corn starch, gelatin and alginic acid; lubricants such as magnesium stearate; sweeteners such as sucrose, lactose or saccharin; flavoring agents such as peppermint, Gaultheria adenothrix oil and cherry. When the unit dosage form is a capsule, a liquid carrier, such as oil, can also be included in the above ingredients. Sterile composites for injections can be formulated following normal drug implementations using vehicles such as distilled water used for injections.

Physiological saline, glucose, and other isotonic liquids including adjuvants, such as D-sorbitol, D-mannnose, D-mannitol, and sodium chloride, can be used as aqueous solutions for injections. These can be used in conjunction with suitable solubilizers, such as alcohol, specifically ethanol, polyalcohols such as propylene glycol and polyethylene glycol, non-ionic surfactants, such as Polysorbate 80 (TM) and HCO-50.

Sesame oil or Soy-bean oil can be used as a oleaginous liquid and may be used in conjunction with benzyl benzoate or benzyl alcohol as a solubilizer; may be formulated with a buffer such as phosphate buffer and sodium acetate buffer; a pain-killer such as procaine hydrochloride; a stabilizer such as benzyl alcohol, phenol; and an anti-oxidant. The prepared injection is filled into a suitable ampule.

Methods well known to one skilled in the art may be used to administer a pharmaceutical compound to patients, for example as intraarterial, intravenous, subcutaneous injections and also as intranasal, transbronchial, intramuscular, percutaneous, or oral administrations. The dosage varies according to the body-weight and age of a patient, and the administration method, but one skilled in the art can suitably select the dosage. If said compound can be encoded by a DNA, the DNA can be inserted into a vector for gene therapy to perform the therapy. The dosage and method of administration vary according to the body-weight, age, and symptoms of a patient, but one skilled in the art can select them suitably.

The dose of the protein of the invention may vary depending on the subject, target organ, symptoms, and administration method, but may be, in general, about 100 μg to 20 mg per day for a normal adult (body weight: 60 kg).

Although varying according to the symptoms, the dose of a compound that binds to a protein of this invention or a compound that inhibits the activity of the protein are generally in the range of about 0.1 to 100 mg, preferably about 1.0 to 50 mg, and more preferably about 1.0 to 20 mg per day for adults (body weight: 60 kg) in the case of an oral administration.

Although varying according to the subject, target organ, symptoms, and method of administration, a single dose of a compound for parenteral administration is preferably, for example, when administered intravenously to normal adults (60 kg body weight) in the form of injection, in the range of about 0.01 to 30 mg, preferably about 0.1 to 20 mg, and more preferably about 0.1 to 10 mg per day. Doses converted to 60 kg body weight or per body surface area can be administered to other animals.

All publications and patents cited herein are incorporated by reference in their entirety.

### DESCRIPTION OF DRAWINGS

FIG. 1 shows an alignment of the amino acid sequences of human DEC2a (hDEC2a) and DEC1.

FIG. 2 shows an alignment of the amino acid sequences of human DEC2a (hDEC2a) and human DEC2b (hDEC2b).

FIG. 3 shows an alignment of the amino acid sequences of human DEC2a (hDEC2a) and mouse DEC2a (mDEC2a).

FIG. 4 shows an alignment of the amino acid sequences of human DEC2a (hDEC2a), mouse DEC2a (mDEC2a), and SHARP-1.

### DETAILED DESCRIPTION

The present invention is described in detail hereinafter using examples, but it is not to be construed as being limited to them.

### EXAMPLE 1

### Isolation of Novel DEC2 Genes Belonging to the DEC1 Subfamily

Using the full-length nucleotide sequence of DEC1 cDNA coding region, the human EST database was searched for cDNA fragments having similarity to DEC1. The full-length cDNA nucleotide sequence of one cDNA fragment selected from the several cDNA fragments searched was determined according the 3′-RACE and 5′-RACE method.

1 μg cDNA synthesized from human skeletal muscle mRNA using SuperScript Preamplification System (Gibco BRL) was used as the template in the first phase PCR. PCR was performed using LA taq (TaKaRa), with a full volume of 50 μl containing 400 μM of each dNTP and 0.2 μM of each primer under the following conditions: 94° C. for 1 min, followed by 30 cycles of 98° C. for 20 sec and 68° C. for 3 min, and finally, 72° C. for 5 min. Thereafter, 5 μl of 1/25 diluents of the reaction solution was used as the template, and second phase PCR was conducted under the same condition as in the first phase. Primers used were as follows: ACT2-5′ (5′-CTATTCGATGATGAAGATACCCCACCAAACCC-3′/SEQ ID NO:3) and 3′006A (5′-GCAAGTGGTTGATCAGCTGGACACA-3′/SEQ ID NO:4) for the first phase of 5′-RACE; ACT2-B (5′-GCTTACCCATACGATGTTCCA-3′/SEQ ID NO:5) and 5′006A (5′-TGGAACGCATCCAAGTCGGACTGAAT-3′/SEQ ID NO:6) for the second phase of 5′-RACE; 065′S2 (5′-TTGAACATGGACGAAGGAATTCC-3′/SEQ ID NO:7) and ACT2-3′ (5′-GAGATGGTGCACGATGCACAGTTGAAGTGAAC-3′/SEQ ID NO:8) for the first phase of 3′-RACE; and 5′006S (5′-ATTCAGTCCGACTTGGATGCGTTCCA-3′/SEQ ID NO:9) and ACT2-3′2 (5′-GCGGGGTTTTTCAGTATCTACGA-3′/SEQ ID NO:10) for the second phase of 3′-RACE.

The PCR products of the second phase were separated by 1% agarose gel electrophoresis, bands were cut out from the gel, and DNA fragments were extracted using GENECLEAN II kit (BIO 101). Obtained DNA fragments were cloned into pGEM-T Easy vector (Promega) by TA cloning. After isolating the clones, plasmids were purified and used for sequencing. Sequences were analyzed using ABI PRISM 310 DNA Analyzer (PE Applied Biosystems) after reacting with BigDye Terminator Cycle Sequencing Ready Reaction (PE Applied Biosystems). Clones obtained from at least three independent PCRs were sequenced and the nucleotide sequences were determined.

Two DEC1 subfamily genes thus identified were named DEC2a and DEC2b. The determined nucleotide sequence of DEC2a cDNA and the amino acid sequence of the protein encoded by the cDNA are shown in SEQ ID NOs:1 and 2, respectively. Furthermore, the determined nucleotide sequence of human DEC2b and amino acid sequence of the protein encoded by the cDNA are shown in SEQ ID NOs:11 and 12, respectively.

Then, the present inventors isolated mouse cDNA corresponding to the isolated human cDNA. First, the mouse EST database was searched for cDNA fragments having similarity to human DEC2a and human DEC2b cDNA using the full-length nucleotide sequences of human DEC2a and 2b cDNA. Then, cDNA fragments, which were presumed to contain the upstream region of the protein translation initiation site or the downstream region of the protein translation termination site, were selected from the searched cDNA fragments, and primers were designed to recover the full-length coding region by PCR. Designed primer sequences were as follows: 5′-AAAATCTCTCCAGGCGACCGT-3′ (SEQ ID NO:15) and 5′-AGCCTGTCGAGCATCGCTTA-3′ (SEQ ID NO:16). Using this primer pair and the cDNA prepared from mouse skeletal muscle mRNA as the template, PCR was conducted under the same conditions as described above. As a result of nucleotide sequence determination of the amplified cDNA, it was revealed that a mouse cDNA with a similar sequence to the human DEC2a had been obtained. This clone was named “mouse DEC2a”. The nucleotide sequence of mouse DEC2a and amino acid sequence of the protein encoded by the cDNA are shown in SEQ ID NOs:13 and 14, respectively.

### EXAMPLE 2

### Structural Analysis of DEC2

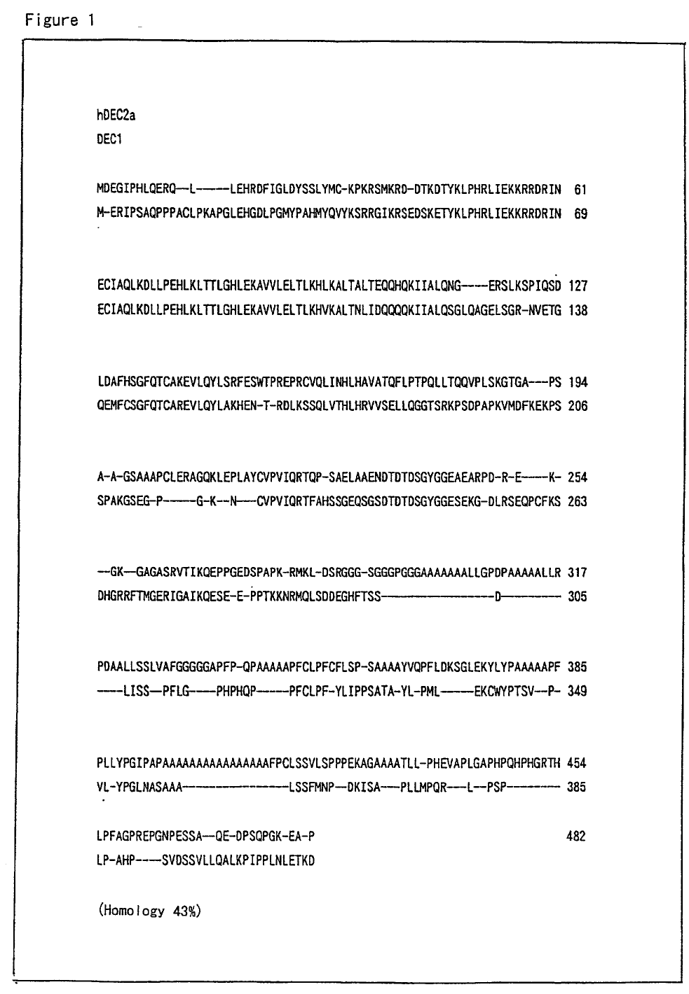

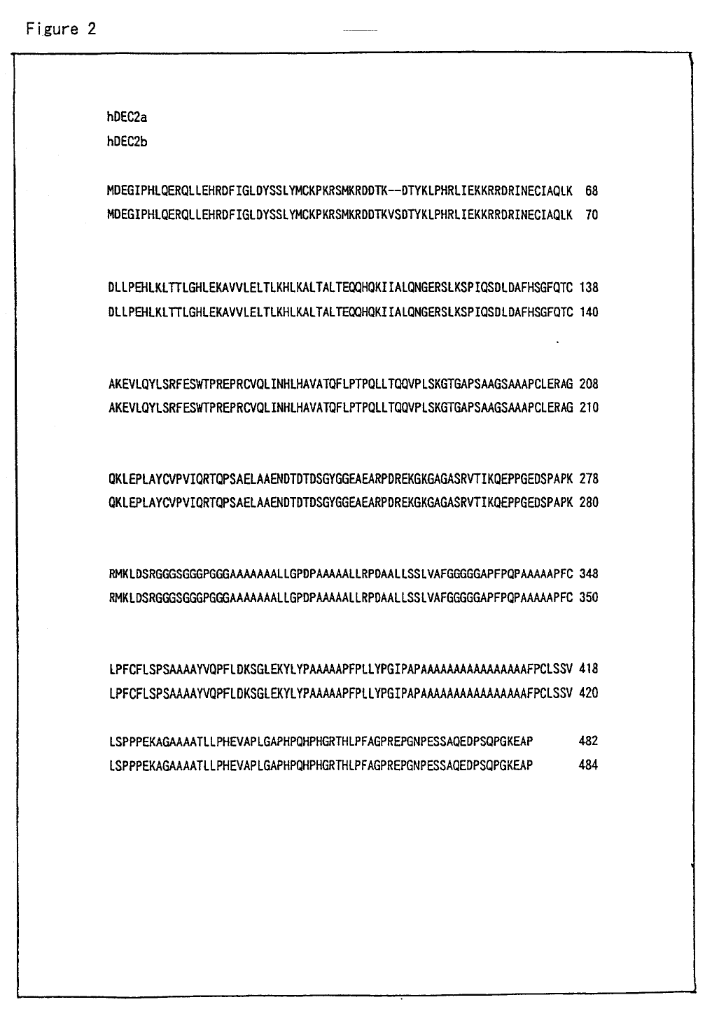

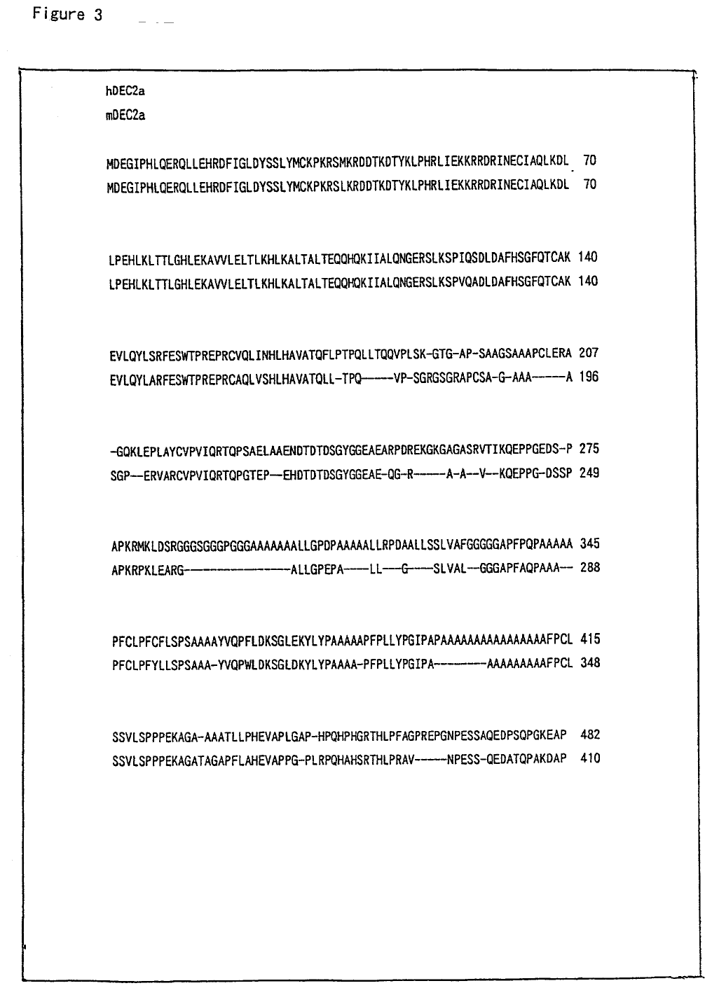

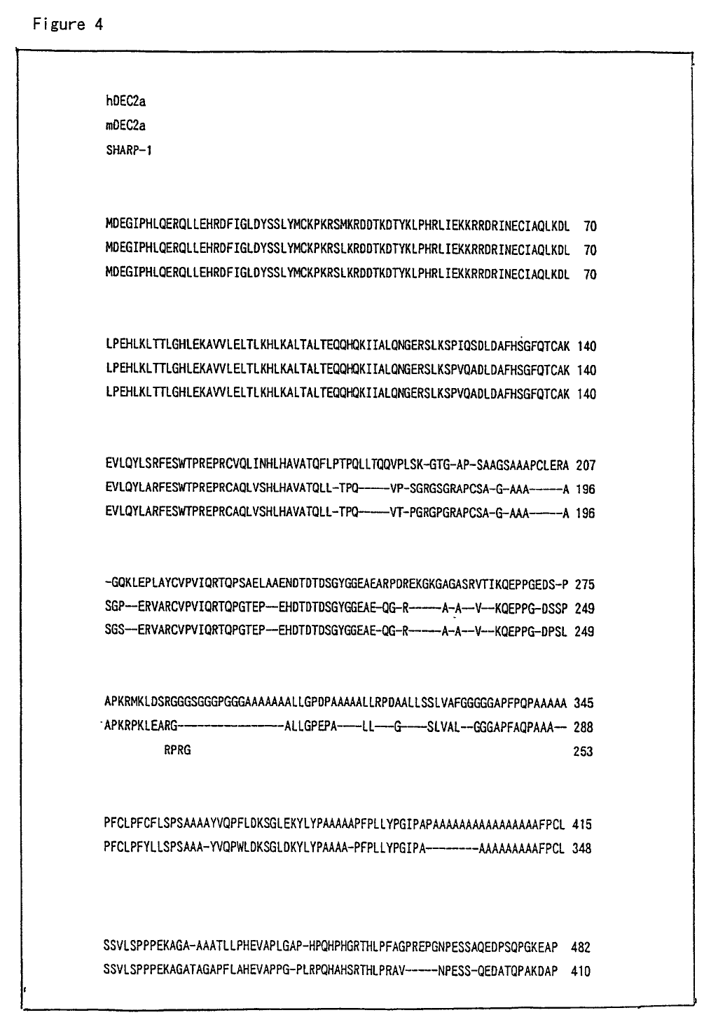

The nucleotide sequence of human DEC2a cDNA had an open reading frame encoding a protein consisting of 482 amino acids. The protein had a basic Helix-Loop-Helix (bHLH) structure, and was supposed to be a bHLH type transcription factor. According to the alignment analysis with the DEC1 protein, an especially high amino acid sequence similarity was detected in the bHLH region (FIG. 1). The structures of human DEC2b and human DEC2a have a high resemblance to each other. and DEC2b had a structure in which 2 amino acids (valine and serine) were inserted immediately before the basic region of the DEC2a bHLH structure. These two are thought to be isoforms of one another (FIG. 2). The nucleotide sequence of mouse DEC2a cDNA had an open reading frame encoding a protein consisting of 410 amino acids, and had a homology of 73% at the amino acid level to human DEC2a (FIG. 3). Mouse DEC2a, human DEC2a and human DEC2b proteins showed the highest homology to the amino acid sequence of rat SHARP protein, and the homology was especially high in the N-terminus half that includes the bHLH region (FIG. 4). It was revealed, however, that the homology between regions of the C-terminus half was relatively low.

### Industrial Applicability

The present invention provides novel bHLH type transcription factors and their genes. Since the genes are thought to be involved in the differentiation and proliferation of cells and tissues, they may be used as factors controlling these cellular functions. Furthermore, they may be used in the purification and cloning of novel factors related to development and cell proliferation, and as tools for developing pharmaceutical agents for various diseases caused by abnormal expression of the genes of the present invention due to abnormal regulation of expression in vivo. Designing drugs and such that target genes of the present invention may enable the development of pharmaceutical agents with novel functional mechanisms.

What is claimed is:

1. An isolated nucleic acid selected from the group consisting of:
(a) a nucleic acid comprising the coding region of the nucleotide sequence of SEQ ID NO:1, 11, or 13;
(b) a nucleic acid encoding a protein comprising the amino acid sequence of SEQ ID NO:2, 12, or 14;
(c) a nucleic acid encoding a protein comprising a modified amino acid sequence of SEQ ID NO:2, 12, or 14, wherein the protein encoded by said nucleic acid retains the biological activity of the protein comprising the amino acid sequence of SEQ ID NO:2, 12, or 14;
(d) a nucleic acid that hybridizes under stringent conditions to a sequence comprising the nucleotide sequence of SEQ ID NO:1, 11, or 13, and encodes a protein that retains the biological activity of the protein comprising the amino acid sequence of SEQ ID NO:2, 12, or 14; and
(e) a nucleic acid encoding a partial peptide of the protein of SEQ ID NO:2, 12, or 14.

2. The nucleic acid of claim 1, wherein the modification referred to in part (c) is a substitution or deletion of less than 20 amino acid residues in the sequence of SEQ ID NO:2, 12, or 14.

3. The nucleic acid of claim 1, wherein the modification referred to in part (c) is a substitution of one or more amino acids in the sequence of SEQ ID NO:2, 12, or 14 with one or more amino acids that allows the properties of a corresponding amino acid side chain to be conserved.

4. The nucleic acid of claim 1, wherein the modification referred to in part (c) is an addition of one or more amino acids to the sequence of SEQ ID NO:2, 12, or 14 that results in a fusion protein.

5. A vector into which the nucleic acid of claim 1 is inserted.

6. A transformant carrying the nucleic acid of claim 1.

7. A substantially pure protein or peptide encoded by the nucleic acid of claim 1.

8. A method for producing a protein or peptide encoded by the nucleic acid of claim 1, comprising the steps of:
(a) culturing a transformant carrying the nucleic acid of claim 1 or a vector into which the nucleic acid of claim 1 is inserted;
(b) allowing the transformant to express the protein or peptide; and
(c) recovering the expressed protein or peptide from the transformant or culture supernatant.

9. An isolated nucleic acid comprising at least 15 nucleotides, wherein the nucleic acid is complementary to a nucleotide sequence comprising the sequence of SEQ ID NO:1, 11, or 13, or to the complementary strand thereof.

10. The nucleic acid of claim 9, wherein the nucleic acid is completely complementary to a continuous region of at least 15 nucleotides in the sequence of SEQ ID NO:1, 11, or 13, or has a homology of at least 70% to the sequence of SEQ ID NO:1, 11, or 13.

11. A method of screening for a compound that binds to the protein or peptide of claim 7, comprising the steps of:
(a) contacting a test sample containing at least one compound with said protein or partial peptide;
(b) detecting the binding activity of said protein or partial peptide with a compound in the test sample; and
(c) selecting a compound that has a binding activity to said protein or partial peptide.

12. A compound that binds to the protein or peptide of claim 7.

13. The compound of claim 12, wherein said compound is an antibody.

14. The compound of claim 12, wherein said compound is isolated by a method comprising the steps of:
(a) contacting a test sample containing at least one compound with said protein or partial peptide;
(b) detecting the binding activity of said protein or partial peptide with a compound in the test sample; and
(c) selecting a compound that has a binding activity to said protein or partial peptide.

15. A transformant carrying the vector of claim 5.


SEQUENCE LISTING (18 sequences)

SEQ ID NO: 1 - DNA, 3641 bp, Homo sapiens
  CDS (135)...(1580)
ctgcactgaagagggagagcgagagagagactggagacgcacagatccccccaaggtctcccaagcctaccgtcccacagattattgtacagagccccaaaaatcgaaacagaggaaacgaacagcagttgaacatggacgaaggaattcctcatttgcaagagagacagttactggaacatagagattttataggactggactattcctctttgtatatgtgtaaacccaaaaggagcatgaaacgagacgacaccaaggatacctacaaattaccgcacagattaatagaaaagaaaagaagagaccgaattaatgaatgcattgctcagctgaaagatttactgcctgaacatctgaaattgacaactctgggacatctggagaaagctgtagtcttggaattaactttgaaacacttaaaagctttaaccgccttaaccgagcaacagcatcagaagataattgctttacagaatggggagcgatctctgaaatcgcccattcagtccgacttggatgcgttccactcgggatttcaaacatgcgccaaagaagtcttgcaatacctctcccggtttgagagctggacacccagggagccgcggtgtgtccagctgatcaaccacttgcacgccgtggccacccagttcttgcccaccccgcagctgttgactcaacaggtccctctgagcaaaggcaccggcgctccctcggccgccgggtccgcggccgccccctgcctggagcgcgcggggcagaagctggagcccctcgcctactgcgtgcccgtcatccagcggactcagcccagcgccgagctcgccgccgagaacgacacggacaccgacagcggctacggcggcgaagccgaggcccggccggaccgcgagaaaggcaaaggcgcgggggcgagccgcgtcaccatcaagcaggagcctcccggggaggactcgccggcgcccaagaggatgaagctggattcccgcggcggcggcagcggcggcggcccggggggcggcgcggcggcggcggcagccgcgcttctggggcccgaccctgccgccgcggccgcgctgctgagacccgacgccgccctgctcagctcgctggtggcgttcggcggaggcggaggcgcgcccttcccgcagcccgcggccgccgcggcccccttctgcctgcccttctgcttcctctcgccttctgcagctgccgcctacgtgcagcccttcctggacaagagcggcctggagaagtatctgtacccggcggcggctgccgccccgttcccgctgctataccccggcatccccgccccggcggcagccgcggcagccgccgccgccgctgccgccgccgccgccgcgttcccctgcctgtcctcggtgttgtcgccccctcccgagaaggcgggcgccgccgccgcgaccctcctgccgcacgaggtggcgccccttggggcgccgcacccccagcacccgcacggccgcacccacctgcccttcgccgggccccgcgagccggggaacccggagagctctgctcaggaagatccctcgcagccaggaaaggaagctccctgaatccttgcgtcccgaaggacggaggttcaagcagagtgagaagttaaaatacccttaaggaggttcaagcagagtgagaagttaaaatacccttaaggtctttaagggaggaagtgtaatagatgcacgacaggcataaacaagaacaacaaaacaggtgttatgtgtacattcggagttcctgttttgctcatcccgcaccaccccaccctccacacactaacatccctttcttccccccaccagctgtaaaagatcctatgcgaaagacactggctcttttttttaatcccccaaataaattttgcccccttttaggccatgttccattatctcttaaaattggaacctaattcgagaggaagtaagaagggtctgttctgtggctgagctaggtgaaccccggggtaggggaaagatgttaacacctttgacgtctttggagttgacatggaacagcaggtagttgttatgtagagctagttctcaaagctgccctgcctgttttaggaggcgttccacaaacagattgaggctctttttagaattgaatttactcttcagtattttctaatgttcagctttctaaaaggcatatatttttcaaagaagtgaggatgcagtttctcacgttgcaacctattctgaagtggtttaaatggtatctcttagtaacttgcactcgttaaagaaacacggagctgggccatcgtcagaactaagtcagggaaggagatggatgagaaggccagaatcattcctagtacatttgctaacactttattgagaaattgaccatgaattaatggactcatcttaatttcttctaagtccatatatagatagatatctatctgtacagatttctatttatccatagataggtatctatacatacacatctcaagtgcatctattcccactctcattaatccatcatgttcctaaatttttgtaatcttactgtaaaaaaaagtgcactgaacttcaaaacaaaacaaaaaacaacaacaacaaaaaacaagtccaaactgatatatcctatattctgttaaaattcaaaagtgaacgaaagcatttaactggccagttttgattgcaaatgctgtaaagatatagaatgaagtcctgtgaggccttcctatctccaagtctatgtattttctggagaccaaaccagataccagataatcacaaagaaagcttttttaataaggcttaaaccaagaccttgtctagatatttttagtttgttgccaaggtagcactgtgagaaatctcacttggatgttatgtaaggggtgagacacaacagtctgactatgagtgaggaaaatatctgggtcttttcgtcagtttggtgcatttgctgctgctgttgctactgtttgcctcaaacgctgtgtttaaacaacgttaaactcttagcctacaaggtggctcttatgtacatagttgttaatacatccaattaatgatgtctgacatgctatttttgtagggagaaaatatgtgctaatgatattttgagttaaaatatcttttggggaggatttgctgaaaagttgcacttttgttacaatgcttatgcttggtacaagcttatgctgtcttaaattattttaaaaaaattaaatactgtctgtgagaaaccagctggtttagaaaagtttagtatgtgacgataaactagaaattacctttatattctagtattttcagcactccataaattctattacctaaatattgccacactattttgtgatttaaaaattcttactaaggaataaaaactttaatatacgatatgatattgtctaataattaaaaaagacataatggatgctcaattagttttaagatatctataactatagggatacaaatcactacagttctcagatttacaccttttttttgtcattggcttgatgtcacacatttccaatctcttgcaagcctccaggctctggctttgtctacctgctcgttcccaatgtatcttaatgaaaagtgcaaaagaaaaacctaccaattaaaaaaaaaaaaaaaaaa

SEQ ID NO: 2 - protein, 482 aa, Homo sapiens
MDEGIPHLQERQLLEHRDFIGLDYSSLYMCKPKRSMKRDDTKDTYKLPHRLIEKKRRDRINECIAQLKDLLPEHLKLTTLGHLEKAVVLELTLKHLKALTALTEQQHQKIIALQNGERSLKSPIQSDLDAFHSGFQTCAKEVLQYLSRFESWTPREPRCVQLINHLHAVATQFLPTPQLLTQQVPLSKGTGAPSAAGSAAAPCLERAGQKLEPLAYCVPVIQRTQPSAELAAENDTDTDSGYGGEAEARPDREKGKGAGASRVTIKQEPPGEDSPAPKRMKLDSRGGGSGGGPGGGAAAAAAALLGPDPAAAAALLRPDAALLSSLVAFGGGGGAPFPQPAAAAAPFCLPFCFLSPSAAAAYVQPFLDKSGLEKYLYPAAAAAPFPLLYPGIPAPAAAAAAAAAAAAAAAAFPCLSSVLSPPPEKAGAAAATLLPHEVAPLGAPHPQHPHGRTHLPFAGPREPGNPESSAQEDPSQPGKEAP

SEQ ID NO: 3 - DNA, 32 bp, Artificial Sequence
  Artificially Synthesized Primer Sequence
ctattcgatgatgaagataccccaccaaaccc

SEQ ID NO: 4 - DNA, 25 bp, Artificial Sequence
  Artificially Synthesized Primer Sequence
gcaagtggttgatcagctggacaca

SEQ ID NO: 5 - DNA, 21 bp, Artificial Sequence
  Artificially Synthesized Primer Sequence
gcttacccatacgatgttcca

SEQ ID NO: 6 - DNA, 26 bp, Artificial Sequence
  Artificially Synthesized Primer Sequence
tggaacgcatccaagtcggactgaat

SEQ ID NO: 7 - DNA, 23 bp, Artificial Sequence
  Artificially Synthesized Primer Sequence
ttgaacatggacgaaggaattcc

SEQ ID NO: 8 - DNA, 32 bp, Artificial Sequence
  Artificially Synthesized Primer Sequence
gagatggtgcacgatgcacagttgaagtgaac

SEQ ID NO: 9 - DNA, 26 bp, Artificial Sequence
  Artificially Synthesized Primer Sequence
attcagtccgacttggatgcgttcca

SEQ ID NO: 10 - DNA, 23 bp, Artificial Sequence
  Artificially Synthesized Primer Sequence
gcggggtttttcagtatctacga

SEQ ID NO: 11 - DNA, 1511 bp, Homo sapiens
  CDS (2)...(1453)
catggacgaaggaattcctcatttgcaagagagacagttactggaacatagagattttataggactggactattcctctttgtatatgtgtaaacccaaaaggagcatgaaacgagacgacaccaaggtaagtgatacctacaaattaccgcacagattaatagaaaagaaaagaagagaccgaattaatgaatgcattgctcagctgaaagatttactgcctgaacatctgaaattgacaactctgggacatctggagaaagctgtagtcttggaattaactttgaaacacttaaaagctttaaccgccttaaccgagcaacagcatcagaagataattgctttacagaatggggagcgatctctgaaatcgcccattcagtccgacttggatgcgttccactcgggatttcaaacatgcgccaaagaagtcttgcaatacctctcccggtttgagagctggacacccagggagccgcggtgtgtccagctgatcaaccacttgcacgccgtggccacccagttcttgcccaccccgcagctgttgactcaacaggtccctctgagcaaaggcaccggcgctccctcggccgccgggtccgcggccgccccctgcctggagcgcgcggggcagaagctggagcccctcgcctactgcgtgcccgtcatccagcggactcagcccagcgccgagctcgccgccgagaacgacacggacaccgacagcggctacggcggcgaagccgaggcccggccggaccgcgagaaaggcaaaggcgcgggggcgagccgcgtcaccatcaagcaggagcctcccggggaggactcgccggcgcccaagaggatgaagctggattcccgcggcggcggcagcggcggcggcccggggggcggcgcggcggcggcggcagccgcgcttctggggcccgaccctgccgccgcggccgcgctgctgagacccgacgccgccctgctcagctcgctggtggcgttcggcggaggcggaggcgcgcccttcccgcagcccgcggccgccgcggcccccttctgcctgcccttctgcttcctctcgccttctgcagctgccgcctacgtgcagcccttcctggacaagagcggcctggagaagtatctgtacccggcggcggctgccgccccgttcccgctgctataccccggcatccccgccccggcggcagccgcggcagccgccgccgccgctgccgccgccgccgccgcgttcccctgcctgtcctcggtgttgtcgccccctcccgagaaggcgggcgccgccgccgcgaccctcctgccgcacgaggtggcgccccttggggcgccgcacccccagcacccgcacggccgcacccacctgcccttcgccgggccccgcgagccggggaacccggagagctctgctcaggaagatccctcgcagccaggaaaggaagctccctgaatccttgcgtcccgaaggacggaggttcaagcagagtgagaagttaaaataccct

SEQ ID NO: 12 - protein, 484 aa, Homo sapiens
MDEGIPHLQERQLLEHRDFIGLDYSSLYMCKPKRSMKRDDTKVSDTYKLPHRLIEKKRRDRINECIAQLKDLLPEHLKLTTLGHLEKAVVLELTLKHLKALTALTEQQHQKIIALQNGERSLKSPIQSDLDAFHSGFQTCAKEVLQYLSRFESWTPREPRCVQLINHLHAVATQFLPTPQLLTQQVPLSKGTGAPSAAGSAAAPCLERAGQKLEPLAYCVPVIQRTQPSAELAAENDTDTDSGYGGEAEARPDREKGKGAGASRVTIKQEPPGEDSPAPKRMKLDSRGGGSGGGPGGGAAAAAAALLGPDPAAAAALLRPDAALLSSLVAFGGGGGAPFPQPAAAAAPFCLPFCFLSPSAAAAYVQPFLDKSGLEKYLYPAAAAAPFPLLYPGIPAPAAAAAAAAAAAAAAAAFPCLSSVLSPPPEKAGAAAATLLPHEVAPLGAPHPQHPHGRTHLPFAGPREPGNPESSAQEDPSQPGKEAP

SEQ ID NO: 13 - DNA, 1421 bp, Mus musculus
  CDS (74)...(1303)
aaaatctctccaggcgaccgtcccacagattacaggacagaaacctccaaatcgaaacggacagccattgaacatggacgaaggaatccctcatttgcaagagagacagttactggaacatagggattttataggactggactattcctctttgtatatgtgtaaacccaaaaggagcttgaagcgagacgataccaaggatacctacaagttaccgcacagattaatagaaaagaagagacgagaccgaattaatgaatgcattgctcagctgaaagatttactgcccgaacatctgaaattgacaacactggggcatttggagaaagcagtagtcttggaattaactttaaagcacttgaaagcgctaacagccttaactgagcagcagcatcagaagataattgctttacagaatggggagcgctctctgaaatcgccggtccaggccgacttggatgcgttccactcggggtttcaaacctgcgccaaagaagtcttgcaatacctcgcgcgctttgagagctggacacccagggagccgcgctgcgcacagctcgtcagccacctgcatgccgtggccacccagctcctgacgccacaggtgccctccggcaggggctctgggcgcgcgccctgcagcgcgggggctgcggccgcctcgggtcccgagcgcgtcgcccgctgcgtgccggtcatccagcggactcagcccggcacggagccggaacacgacacggacaccgacagcggctacggaggcgaggcggagcagggccgcgcggccgtcaagcaggagccacccggggactcgtcgcctgcgcccaagaggccgaagctggaggcgcgcggcgcgctcctgggcccggagcccgcgctgctcggctcgctcgtggcgcttggcgggggcgcgcccttcgcgcagcccgctgccgcgcccttctgcctgcccttctatctgctgtcgccgtccgccgccgcctacgtacagccctggctagacaagagcggcctggacaagtatctgtaccccgcggcggccgcgcccttcccgctgctgtatcccggcatcccagcagcggccgccgctgctgctgccgccgctttcccttgcttgtcgtccgtgctgtcgccacccccggagaaggccggcgcgaccgccggtgccccgttcctggcgcacgaggtggcgcccccggggccgctgcgcccccagcacgcgcatagccgcacccacctgccgcgcgctgtgaacccggagagctctcaggaagatgccacgcagccggccaaggacgccccctgaacccagcattccttccagaacagggcagggggctcccgaggagtcgccgggtttccaagttcaaacgtcctctaaagcgtgccagggaggaagagtaagcgatgctcgacaggct

SEQ ID NO: 14 - protein, 410 aa, Mus musculus
MDEGIPHLQERQLLEHRDFIGLDYSSLYMCKPKRSLKRDDTKDTYKLPHRLIEKKRRDRINECIAQLKDLLPEHLKLTTLGHLEKAVVLELTLKHLKALTALTEQQHQKIIALQNGERSLKSPVQADLDAFHSGFQTCAKEVLQYLARFESWTPREPRCAQLVSHLHAVATQLLTPQVPSGRGSGRAPCSAGAAAASGPERVARCVPVIQRTQPGTEPEHDTDTDSGYGGEAEQGRAAVKQEPPGDSSPAPKRPKLEARGALLGPEPALLGSLVALGGGAPFAQPAAAPFCLPFYLLSPSAAAYVQPWLDKSGLDKYLYPAAAAPFPLLYPGIPAAAAAAAAAAFPCLSSVLSPPPEKAGATAGAPFLAHEVAPPGPLRPQHAHSRTHLPRAVNPESSQEDATQPAKDAP

SEQ ID NO: 15 - DNA, 21 bp, Artificial Sequence
  Artificially Synthesized Primer Sequence
aaaatctctccaggcgaccgt

SEQ ID NO: 16 - DNA, 20 bp, Artificial Sequence
  Artificially Synthesized Primer Sequence
agcctgtcgagcatcgctta

SEQ ID NO: 17 - protein, 412 aa, Homo sapiens
MERIPSAQPPPACLPKAPGLEHGDLPGMYPAHMYQVYKSRRGIKRSEDSKETYKLPHRLIEKKRRDRINECIAQLKDLLPEHLKLTTLGHLEKAVVLELTLKHVKALTNLIDQQQQKIIALQSGLQAGELSGRNVETGQEMFCSGFQTCAREVLQYLAKHENTRDLKSSQLVTHLHRVVSELLQGGTSRKPSDPAPKVMDFKEKPSSPAKGSEGPGKNCVPVIQRTFAHSSGEQSGSDTDTDSGYGGESEKGDLRSEQPCFKSDHGRRFTMGERIGAIKQESEEPPTKKNRMQLSDDEGHFTSSDLISSPFLGPHPHQPPFCLPFYLIPPSATAYLPMLEKCWYPTSVPVLYPGLNASAAALSSFMNPDKISAPLLMPQRLPSPLPAHPSVDSSVLLQALKPIPPLNLETKD

SEQ ID NO: 18 - protein, 253 aa, Rattus norvegicus
MDEGIPHLQERQLLEHRDFIGLDYSSLYMCKPKRSLKRDDTKDTYKLPHRLIEKKRRDRINECIAQLKDLLPEHLKLTTLGHLEKAVVLELTLKHLKALTALTEQQHQKIIALQNGERSLKSPVQADLDAFHSGFQTCAKEVLQYLARFESWTPREPRCAQLVSHLHAVATQLLTPQVTPGRGPGRAPCSAGAAAASGSERVARCVPVIQRTQPGTEPEHDTDTDSGYGGEAEQGRAAVKQEPPGDPSLRPRG